SEQUENCE LISTING (37 sequences)

SEQ ID NO: 1 - DNA, 20 bp, Artificial Sequence
  Primer
aggaattatgaaaccacttg

SEQ ID NO: 2 - DNA, 20 bp, Artificial Sequence
  Primer
gacaacagttgtatcagacc

SEQ ID NO: 3 - DNA, 21 bp, Artificial Sequence
  Primer
caaggagaggtcgtgtcttcg

SEQ ID NO: 4 - DNA, 27 bp, Artificial Sequence
  Primer
ggatcttgttacatgctcactcatatc

SEQ ID NO: 5 - DNA, 20 bp, Artificial Sequence
  Primer
ccagattaagaataacgttc

SEQ ID NO: 6 - DNA, 20 bp, Artificial Sequence
  Primer
agaggaaatgaccaagagtc

SEQ ID NO: 7 - DNA, 21 bp, Artificial Sequence
  Primer
acaagactagcttatgtgtgg

SEQ ID NO: 8 - DNA, 21 bp, Artificial Sequence
  Primer
ttgagcaagaatcttgacttc

SEQ ID NO: 9 - DNA, 23 bp, Artificial Sequence
  Primer
gggagtattgacagtggcaattt

SEQ ID NO: 10 - DNA, 22 bp, Artificial Sequence
  Primer
tgttctcaatgtagcgcctttc

SEQ ID NO: 11 - DNA, 29 bp, Artificial Sequence
  Probe
ccacctgtagctataccagccagactccc

SEQ ID NO: 12 - DNA, 19 bp, Artificial Sequence
  Primer
gcagagtcccctccctgac

SEQ ID NO: 13 - DNA, 23 bp, Artificial Sequence
  Primer
acaggaactggctctgcttaaga

SEQ ID NO: 14 - DNA, 26 bp, Artificial Sequence
  Probe
tcaggaccatctccaggtgcatcctc

SEQ ID NO: 15 - DNA, 28 bp, Artificial Sequence
  Primer
ccagagtctcatgttaaaatcacttaca

SEQ ID NO: 16 - DNA, 30 bp, Artificial Sequence
  Primer
gaaacacttcctctctttctttaagtacaa

SEQ ID NO: 17 - DNA, 28 bp, Artificial Sequence
  Probe
acccagaaagaccaccactttgcaggta

SEQ ID NO: 18 - DNA, 2640 bp, Homo sapiens
catctcgcacttccaactgccctgtaaccaccaactgcccttattccggctgggacccaggacttcaagccatgtgggtcttgtttctgctcagcgggctcggcgggctgcggatggacagtaattttgatagtttacctgtgcaaattacagttccggagaaaatacggtcaataataaaggaaggaattgaatcgcaggcatcctacaaaattgtaattgaagggaaaccatatactgtgaatttaatgcaaaaaaactttttaccccataattttagagtttacagttatagtggcacaggaattatgaaaccacttgaccaagattttcagaatttctgccactaccaagggtatattgaaggttatccaaaatctgtggtgatggttagcacatgtactggactcaggggcgtactacagtttgaaaatgttagttatggaatagaacccctggagtcttcagttggctttgaacatgtaatttaccaagtaaaacataagaaagcagatgtttccttatataatgagaaggatattgaatcaagagatctgtcctttaaattacaaagcgcagagccacagcaagattttgcaaagtatatagaaatgcatgttatagttgaaaaacaattgtataatcatatggggtctgatacaactgttgtcgctcaaaaagttttccagttgattggattgacgaatgctatttttgtttcatttaatattacaattattctgtcttcattggagctttggatagatgaaaataaaattgcaaccactggagaagctaatgagttattacacacatttttaagatggaaaacatcttatcttgttttacgtcctcatgatgtggcatttttacttgtttacagagaaaagtcaaattatgttggtgcaacctttcaagggaagatgtgtgatgcaaactatgcaggaggtgttgttctgcaccccagaaccataagtctggaatcacttgcagttattttagctcaattattgagccttagtatggggatcacttatgatgacattaacaaatgccagtgctcaggagctgtctgcattatgaatccagaagcaattcatttcagtggtgtgaagatctttagtaactgcagcttcgaagactttgcacattttatttcaaagcagaagtcccagtgtcttcacaatcagcctcgcttagatccttttttcaaacagcaagcagtgtgtggtaatgcaaagctggaagcaggagaggagtgtgactgtgggactgaacaggattgtgcccttattggagaaacatgctgtgatattgccacatgtagatttaaagccggttcaaactgtgctgaaggaccatgctgcgaaaactgtctatttatgtcaaaagaaagaatgtgtaggccttcctttgaagaatgcgacctccctgaatattgcaatggatcatctgcatcatgcccagaaaaccactatgttcagactgggcatccgtgtggactgaatcaatggatctgtatagatggagtttgtatgagtggggataaacaatgtacagacacatttggcaaagaagtagagtttggcccttcagaatgttattctcaccttaattcaaagactgatgtatctggaaactgtggtataagtgattcaggatacacacagtgtgaagctgacaatctgcagtgcggaaaattaatatgtaaatatgtaggtaaatttttattacaaattccaagagccactattatttatgccaacataagtggacatctctgcattgctgtggaatttgccagtgatcatgcagacagccaaaagatgtggataaaagatggaacttcttgtggttcaaataaggtttgcaggaatcaaagatgtgtgagttcttcatacttgggttatgattgtactactgacaaatgcaatgatagaggtgtatgcaataacaaaaagcactgtcactgtagtgcttcatatttacctccagattgctcagttcaatcagatctatggcctggtgggagtattgacagtggcaattttccacctgtagctataccagccagactccctgaaaggcgctacattgagaacatttaccattccaaaccaatgagatggccatttttcttattcattcctttctttattattttctgtgtactgattgctataatggtgaaagttaatttccaaaggaaaaaatggagaactgaggactattcaagcgatgagcaacctgaaagtgagagtgaacctaaagggtagtctggacaacagagatgccatgatatcacttcttctagagtaattatctgtgatggatggacacaaaaaaatggaaagaaaagaatgtacattacctggtttcctgggattcaaacctgcatattgtgattttaatttgaccagaaaatatgatatatatgtataatttcacagataatttacttatttaaaaatgcatgataatgagttttacattacaaatttctgtttttttaaagttatcttacgctatttctgttggttagtagacactaattctgtcagtaggggcatggtataaggaaatatcataatgtaatgaggtggtactatgattaaaagccactgttacatttcaaaaaaa

SEQ ID NO: 19 - protein, 734 aa, Homo sapiens
MWVLFLLSGLGGLRMDSNFDSLPVQITVPEKIRSIIKEGIESQASYKIVIEGKPYTVNLMQKNFLPHNFRVYSYSGTGIMKPLDQDFQNFCHYQGYIEGYPKSVVMVSTCTGLRGVLQFENVSYGIEPLESSVGFEHVIYQVKHKKADVSLYNEKDIESRDLSFKLQSAEPQQDFAKYIEMHVIVEKQLYNHMGSDTTVVAQKVFQLIGLTNAIFVSFNITIILSSLELWIDENKIATTGEANELLHTFLRWKTSYLVLRPHDVAFLLVYREKSNYVGATFQGKMCDANYAGGVVLHPRTISLESLAVILAQLLSLSMGITYDDINKCQCSGAVCIMNPEAIHFSGVKIFSNCSFEDFAHFISKQKSQCLHNQPRLDPFFKQQAVCGNAKLEAGEECDCGTEQDCALIGETCCDIATCRFKAGSNCAEGPCCENCLFMSKERMCRPSFEECDLPEYCNGSSASCPENHYVQTGHPCGLNQWICIDGVCMSGDKQCTDTFGKEVEFGPSECYSHLNSKTDVSGNCGISDSGYTQCEADNLQCGKLICKYVGKFLLQIPRATIIYANISGHLCIAVEFASDHADSQKMWIKDGTSCGSNKVCRNQRCVSSSYLGYDCTTDKCNDRGVCNNKKHCHCSASYLPPDCSVQSDLWPGGSIDSGNFPPVAIPARLPERRYIENIYHSKPMRWPFFLFIPFFIIFCVLIAIMVKVNFQRKKWRTEDYSSDEQPESESEPKG

SEQ ID NO: 20 - DNA, 528 bp, Homo sapiens
ggcacgaggcttcgttctttccgccatcttcgttctttccaacatcttcgttctttctcactgaccgagactcagccgtgagagatatgagtgagcatgtaacaagatcccaatcctcagaaagaggaaatgaccaagagtcttcccagccagttggacctgtgattgtccagcagcccactgaggaaaaacgtcaagaagaggaaccaccaactgataatcagggtattgcacctagtggggagatcaaaaatgaaggagcacctgctgttcaagggactgatgtggaagcttttcaacaggaactggctctgcttaagatagaggatgcacctggagatggtcctgatgtcagggaggggactctgcccacttttgatcccactaaagtgctggaagcaggtgaagggcaactataggtttaaaccaagacaaatgaagactgaaaccaagaatattgttcttatgctggaaatttgactgctaacattctcttaataaagttttacagttttctgcaaaaaaaaa

SEQ ID NO: 21 - protein, 110 aa, Homo sapiens
MSEHVTRSQSSERGNDQESSQPVGPVIVQQPTEEKRQEEEPPTDNQGIAPSGEIKNEGAPAVQGTDVEAFQQELALLKIEDAPGDGPDVREGTLPTFDPTKVLEAGEGQL

SEQ ID NO: 22 - DNA, 877 bp, Homo sapiens
ggcacgaggcttgttcatggcatctttagaaacaaactgcaattttatttcatttccttgtcgttcatacaaagattacaagactagcttatgtgtggactgtgactgttttaaggaaaaatcatgtcctcggctgggttatcaagccaagctatttaaaggtgttttaaaagaaaggatggaaggaagacctcttaggaccactgtgtttttggatacaagtggtacatatccattctgtacctattattttgttctcagtataattgttccagataaaactatgatggatggctcgttttcatttaaattattaaatcagcttgaaatgattgaagagccaaggctttatgaaaagaacaaaccattttataaacttcaagaagtcaagattcttgctcaattttataatgactttgtaaatatttcaagcattggtttgacatatttccagagctcaaatctgcagtgttccacatgcacatacaagatccagagtctcatgttaaaatcacttacatacccagaaagaccaccactttgcaggtataatattgtacttaaagaaagagaggaagtgtttcttaatccaaacacatgtacaccaaagaacacataagatgccttcttccatcaaatgcacttgcttgtgaattaatggacttgtaaatgaaacaatgcaatcagtcttttataatacactgttcaatttgagattcaagtatttctatttcttggaaaaaattttaagaatcaaaaataaagaaaataaaaagtgcatacagttaaacattccaaaaaaaaaaaaaaaaaaaaaaaaaaaattggcggccgcaagcttattccctttagtgagggttaattttagcttggcact

SEQ ID NO: 23 - protein, 205 aa, Homo sapiens
ARGLFMASLETNCNFISFPCRSYKDYKTSLCVDCDCFKEKSCPRLGYQAKLFKGVLKERMEGRPLRTTVFLDTSGTYPFCTYYFVLSIIVPDKTMMDGSFSFKLLNQLEMIEEPRLYEKNKPFYKLQEVKILAQFYNDFVNISSIGLTYFQSSNLQCSTCTYKIQSLMLKSLTYPERPPLCRYNIVLKEREEVFLNPNTCTPKNT

SEQ ID NO: 24 - DNA, 2270 bp, Homo sapiens
gggcacattgcagattgttcgggtaaatttaaaatgctagagcatgccctacgtgatgccaagatggcggagacttgtattgtgaaagaaaagcaagattataagcagaaattgaaggcacttaagattgaagtcaacaaactaaaagaggacctcaatgaaaagacgacagaaaataatgagcaacgagaagagatcattcgcctcaagcaagagaaaagttgcctgcacgatgaattgctttttactgtagagagagaaaagaggaaagatgaattgcttaatattgcgaagtcaaagcaagaacgcacaaattcagaactgcacaatctgagacagatttatgtaaaacaacagagtgatctgcagtttcttaatttcaatgtggaaaattctcaggaattaatacagatgtatgactcaaagatggaggaatcaaaggctctggactccagcagagacatgtgtttatcagaccttgaaaataaccacccaaaagtcgatattaagagggaaaaaaatcagaagtcactgtttaaggaccagaaatttgaagccatgttggttcagcaaaataggtcagacaagagctcttgcgatgaatgcaaagagaagaaacaacagatcgatactgtgtttggggagaaaagtgtaattacgctgtcatccatattcaccaaagacttagtagagaaacacaacctcccttggtctctgggaggaaaaacccagattgaacccgaaaacaaaattacattgtgcaagatccacacaaaatcaccaaaatgtcatggcactggggttcagaacgaaggaaaacaaccctcagaaacacccactttatctgatgagaagcagtggcatgatgtcagtgtttacctgggcctgaccaactgtccaagttcaaaacatccagaaaagctggatgtagaatgtcaagatcagatggaaaggtccgaaatctcatgctgccagaaaaatgaagcctgtctgggcgaaagtggcatgtgtgactccaagtgctgccacccgagtaacttcataattgaagccccaggccacatgtctgacgtggagtggatgagtattttcaagccttccaaaatgcagagaattgtccgcctcaaatctgggtgcacctgttcagaaagcatctgtggcacacaacatgactccccggcaagtgagctaattgccatccaagattcccactctttgggttcttcaaaatctgccttgagagaagatgagacggagtcctcttccaataaaaagaactcacctacgagtttgttaatctacaaagatgcaccagcattcaatgaaaaggcttcaattgtgttaccctcccaggatgatttctcgcccacgagcaagctccagcgtttgctggcggaatctcgtcagatggtgacggacctggagctgaacacactgctgcccatcagccatgagaatctcactggcagtgccacaaatatttctcatctatgtggaaggcagaaagcagacaccaatactgaatgaatacttaaccgtaaaactgaaagaggattctagttcttcataaacggcacttaattccagctgggagcagaactagaaagttaatttttaaacatctacacttcattttcaagttaaccatttttgtgctgaagaaatattttcatgtgtaagaaagtagaccttattgtacatatagaaagttggaattatgctaagaatgaaaaagacttctctgtaaagatacagactacagttaaatgctagagaagctctttaaaaatgtgaatgtcaaatagagaaagaacccctgcatagaaagtgctgttttaactatctgatttttaaaaaatctgtgcatacatttaaattctaaacaatagcttatcagagtcagctcaaaatatatgagaaacagtattctctcatggttttagcttttgactttgctgtgtaaatagacataaggtgctttgatataaaatataaagtgtaactggaaaatagctcgaggtccttctgtcccaagctgagcagagccccatctttctgggtctatattagtcccacctactgacacaaacaaaagcttgctggaagatcgagttttagacgcatttttaaaaatcttaaagactaaaacacttccattttaacttgtaaagtaatttaattttttaaagattatactatatgcctctgtgtcttctctaaaagaatagatcaacttcagtccataaaagatatttttaatattaaagaaa

SEQ ID NO: 25 - protein, 497 aa, Homo sapiens
MLEHALRDAKMAETCIVKEKQDYKQKLKALKIEVNKLKEDLNEKTTENNEQREEIIRLKQEKSCLHDELLFTVEREKRKDELLNIAKSKQERTNSELHNLRQIYVKQQSDLQFLNFNVENSQELIQMYDSKMEESKALDSSRDMCLSDLENNHPKVDIKREKNQKSLFKDQKFEAMLVQQNRSDKSSCDECKEKKQQIDTVFGEKSVITLSSIFTKDLVEKHNLPWSLGGKTQIEPENKITLCKIHTKSPKCHGTGVQNEGKQPSETPTLSDEKQWHDVSVYLGLTNCPSSKHPEKLDVECQDQMERSEISCCQKNEACLGESGMCDSKCCHPSNFIIEAPGHMSDVEWMSIFKPSKMQRIVRLKSGCTCSESICGTQHDSPASELIAIQDSHSLGSSKSALREDETESSSNKKNSPTSLLIYKDAPAFNEKASIVLPSQDDFSPTSKLQRLLAESRQMVTDLELNTLLPISHENLTGSATNISHLCGRQKADTNTE

SEQ ID NO: 26 - DNA, 996 bp, Homo sapiens
gtatgttcctggcagaaaagttgcataacttgggggttaaagacaggaatatggcacaatgcccattgtgggcagtgtccagacagaagaagagactcatatcacctaaatgacaaacccagaatatgtcacaatgtatcctgttgaaaggaccagaaacgaggatgaattgccacatcatctggttctgagttctgagatattcacaagtccccttagaaaacaactcaggcaagagagttacatcacctaggagcccgttccacccttatgtcacagtgcttcatgtgtacaggactaagaagaaagtcacatcacctagatgatagacccagagacacgtcacaaagccttcctgaaagcatggccctggcaaaatagtacaatcacctttgtacctggtctagcaatatgtcattattcaagtgtgcaggtaccaaggagaggagccatactacctatgttatatgccctgtgctatgtcaaaatgccttcttttcagcatggccctggaagaatgtatcatctcacatgtgactggcctaggaagatgtcactatcctgccatgtgtgcagggcccattttagagattagagttatgtcttcttctaagtcatggacccagtgatatatcactatgatgtctgtgagaatgggcaggcaggaatgtaatgtcacttgcattctagatccagtgatgtcacaattcttactgagggcagagcccagtcagaagagtcatatcttttacaggttggcccaagtagatgtcaaaaacccctatggattagatttacaataccacacatctcttgttttcatgtaggagagttgcctgcattcatctgtgatgatgaaagtccttactgtcagccaagtgtgcatatgagactcacaatttcctctgtttgctaaccactattatgacactctctattcaacccaacggttttataaaacatttgtcattgttataaaaaaaaaaaaaaaaaaaa

SEQ ID NO: 27 - protein, 90 aa, Homo sapiens
MIDPETRHKAFLKAWPWQNSTITFVPGLAICHYSSVQVPRRGAILPMLYALCYVKMPSFQHGPGRMYHLTCDWPRKMSLSCHVCRAHFRD

SEQ ID NO: 28 - DNA, 2347 bp, Homo sapiens
gggaggtttggagccctgcataaagagaaggacgggaccacagctgactgctgtgtccccacagatctgggcctcctgctgccaccatggccaaaggtggagaagccctgccacagggcagcccagcaccagtccaggatccccacctcatcaaggtgacagtgaagacgcccaaagacaaggaggatttctcagttacagacacatgcactatccagcagctgaaggaagagatatctcagcgctttaaggcccaccccgatcagcttgttctaatctttgctggcaaaatcctcaaggatcctgactcactggcacagtgtggagtgcgagatggcctcactgtccacctggtcatcaagaggcagcaccgtgccatgggcaatgagtgcccagctgcctctgtccctacccagggcccaagtcctggatcactccctcagccaagctccatttacccagcagatgggccccctgcctttagcttaggtctcctcacaggcctcagtaggctgggcttggcctatcgtggcttccctgaccagccaagctccctgatgcggcagcatgtgtctgtgcctgagtttgtgactcagctcattgatgaccccttcatcccgggtctgctgtccaacacaggcctagtacgccagctggttcttgacaacccccatatgcagcagctgatccagcacaaccctgagattgggcatattcttaacaacccggaaattatgcggcagacactggagtttttacgtaaccctgccatgatgcaggagatgatacgtagccaggaccgggtgctcagtaacttggagagcattcctggtggctacaatgtgctttgcactatgtacacagatattatggacccaatgcttaacgcagtccaggagcagtttggcggcaatccctttgccactgccactactgataatgccaccaccaccaccagccaaccttcaaggatggagaattgtgaccctctccccaacccctggacttccacacatggaggctcaggtagcaggcaaggaaggcaggatggggatcaggatgcacctgacattagaaataggtttccaaactttctgggtattataaggctctatgactatctccagcaattacacgagaacccccagtccctaggaacttatctacaggggactgcatctgccctcagccaaagccaggaaccaccaccatcagtaaacagagttcccccatcgtcaccctcatctcaggagcctgggtcaggccagcctctccccgaggagtcagtagcaatcaagggaaggtcctcctgcccagctttcctgagataccccacagagaacagtactggacaaggtggagaccaagatggtgcagggaaaagctctactggacatagcacaaacttgcctgatcttgtctcggggctgggagattctgccaacagggttccatttgctcccttatctttttcccccacggcagccattcctggaatccctgagcctccctggctgccatccccggcttatccaagatctctgaggccagatggcatgaatccagctccacagttacaggatgagatacaaccacagctgccactgctgatgcaccttcaggcagccatggcaaacccccgtgccctgcaagccctgcggcagattgagcagggtctacaggtcctagctactgaagcacctcgcctcctactctggttcatgccttgcctagcagggacgggtagtgtggcaggaggtatagagtctagagaagatccccttatgtctgaggatcctctcccaaatccacctcctgaggtgttcccagcactggactctgcagagctgggcttcctttcccctccctttctccatatgctgcaagatttagttagtacaaatccccagcagctgcagcctgaggctcactttcaggtgcagctggagcaactgcggtccatgggctttctgaatcgtgaagccaatcttcaggccctcattgctacggggggcgacgtggatgctgctgtggagaagctgagacagtcgtaggagccttattcattcaaaccatacgttttcctctgtgcctttttcccatatcctagttccctagctctcccatttttgaatacagctgcattataaaccaaatttactatgaagtcctttgctgtggaggcaatgttgttccagagtcaacgaggaagactaatggccaaaacatagtggaggtgctgtgtgtgagtcaaccacttgtaccactataccactggggggccccagtctaagctctgcttatgcctatcttgagatgcaattacacccaatttccaatgtgaaaa

SEQ ID NO: 29 - protein, 655 aa, Homo sapiens
MAKGGEALPQGSPAPVQDPHLIKVTVKTPKDKEDFSVTDTCTIQQLKEEISQRFKAHPDQLVLIFAGKILKDPDSLAQCGVRDGLTVHLVIKRQHRAMGNECPAASVPTQGPSPGSLPQPSSIYPADGPPAFSLGLLTGLSRLGLAYRGFPDQPSSLMRQHVSVPEFVTQLIDDPFIPGLLSNTGLVRQLVLDNPHMQQLIQHNPEIGHILNNPEIMRQTLEFLRNPAMMQEMIRSQDRVLSNLESIPGGYNVLCTMYTDIMDPMLNAVQEQFGGNPFATATTDNATTTTSQPSRMENCDPLPNPWTSTHGGSGSRQGRQDGDQDAPDIRNRFPNFLGIIRLYDYLQQLHENPQSLGTYLQGTASALSQSQEPPPSVNRVPPSSPSSQEPGSGQPLPEESVAIKGRSSCPAFLRYPTENSTGQGGDQDGAGKSSTGHSTNLPDLVSGLGDSANRVPFAPLSFSPTAAIPGIPEPPWLPSPAYPRSLRPDGMNPAPQLQDEIQPQLPLLMHLQAAMANPRALQALRQIEQGLQVLATEAPRLLLWFMPCLAGTGSVAGGIESREDPLMSEDPLPNPPPEVFPALDSAELGFLSPPFLHMLQDLVSTNPQQLQPEAHFQVQLEQLRSMGFLNREANLQALIATGGDVDAAVEKLRQS

SEQ ID NO: 30 - DNA, 899 bp, Homo sapiens
ctagagagtatagggcagaaggatggcagatgagtgactccacatccagagctgcctccctttaatccaggatcctgtccttcctgtcctgtaggagtgcctgttgccagtgtggggtgagacaagtttgtcccacagggctgtctgagcagataagattaagggctgggtctgtgctcaattaactcctgtgggcacgggggctgggaagagcaaagtcagcggtgcctacagtcagcaccatgctgggcctgccgtggaagggaggtctgtcctgggcgctgctgctgcttctcttaggctcccagatcctgctgatctatgcctggcatttccacgagcaaagggactgtgatgaacacaatgtcatggctcgttacctccctgccacagtggagtttgctgtccacacattcaaccaacagagcaaggactactatgcctacagactggggcacatcttgaattcctggaaggagcaggtggagtccaagactgtattctcaatggagctactgctggggagaactaggtgtgggaaatttgaagacgacattgacaactgccatttccaagaaagcacagagctgaacaatactttcacctgcttcttcaccatcagcaccaggccctggatgactcagttcagcctcctgaacaagacctgcttggagggattccactgagtgaaacccactcacaggcttgtccatgtgctgctcccacattccgtggacatcagcactactctcctgaggactcttcagtggctgagcagctttggacttgtttgttatcctattttgcatgtgtttgagatctcagatcagtgttttagaaaatccacacatcttgagcctaatcatgtagtgtagatcattaaacatcagcattttaag

SEQ ID NO: 31 - protein, 147 aa, Homo sapiens
MLGLPWKGGLSWALLLLLLGSQILLIYAWHFHEQRDCDEHNVMARYLPATVEFAVHTFNQQSKDYYAYRLGHILNSWKEQVESKTVFSMELLLGRTRCGKFEDDIDNCHFQESTELNNTFTCFFTISTRPWMTQFSLLNKTCLEGFH

SEQ ID NO: 32 - DNA, 2186 bp, Homo sapiens
tggagaggccacagctgctggcttcctgggcttctccaaactcctgtgtgtcgccactgccaccggcagggagccaggagagagacagaaaggggctgagacagaatgatcaaaaggagagcccaccctggtgcgggaggcgacaggaccaggcctcgacggcgccgttccactgagagctggattgaaagatgtctcaacgaaagtgaaaacaaacgttattccagccacacatctctggggaatgtttctaatgatgaaaatgaggaaaaagaaaataatagagcatccaagccccactccactcctgctactctgcaatggctggaggagaactatgagattgcagagggggtctgcatccctcgcagtgccctctatatgcattacctggatttctgcgagaagaatgatacccaacctgtcaatgctgccagctttggaaagatcataaggcagcagtttcctcagttaaccaccagaagactcgggacccgaggacagtcaaagtaccattactatggcattgcagtgaaagaaagctcccaatattatgatgtgatgtattccaagaaaggagctgcctgggtgagtgagacgggcaagaaagaagtgagcaaacagacagtggcatattcaccccggtccaaactcggaacactgctgccagaatttcccaatgtcaaagatctaaatctgccagccagcctgcctgaggagaaggtttctacctttattatgatgtacagaacacactgtcagagaatactggacactgtaataagagccaactttgatgaggttcaaagtttccttctgcacttttggcaaggaatgccgccccacatgctgcctgtgctgggctcctccacggtggtgaacattgtcggcgtgtgtgactccatcctctacaaagctatctccggggtgctgatgcccactgtgctgcaggcattacctgacagcttaactcaggtgattcgaaagtttgccaagcaactggatgagtggctaaaagtggctctccacgacctcccagaaaacttgcgaaacatcaagttcgaattgtcgagaaggttctcccaaattctgagacggcaaacatcactaaatcatctctgccaggcatctcgaacagtgatccacagtgcagacatcacgttccaaatgctggaagactggaggaacgtggacctgaacagcatcaccaagcaaaccctttacaccatggaagactctcgcgatgagcaccggaaactcatcacccaattatatcaggagtttgaccatctcttggaggagcagtctcccatcgagtcctacattgagtggctggataccatggttgaccgctgtgttgtgaaggtggctgccaagagacaagggtccttgaagaaagtggcccagcagttcctcttgatgtggtcctgtttcggcacaagggtgatccgggacatgaccttgcacagcgcccccagcttcgggtcttttcacctaattcacttaatgtttgatgactacgtgctctacctgttagaatctctgcactgtcaggagcgggccaatgagctcatgcgagccatgaagggagaaggaagcactgcagaagtccgagaagagatcatcttgacagaggctgccgcaccaaccccttcaccagtgccatcgttttctccagcaaaatctgccacatctgtggaagtgccacctccctcttcccctgttagcaatccttcccctgagtacactggcctcagcactacaggtaatggaaagtccttcaaaaactttgggtagttaatgtttgaagaaagggctttctgccagcctgggcaacatagtgagacttcatttccacacacacaaaaagccagacatcttggctcacacctgtagtcccagctacttgggaggctgaggtgggagaattgcttgagcccaggagctacgatcgcaccactgcattctagccttagtgatacagtgagaccttgtctcaaaaaaggaaaaacagggctttctggaaaaacattcttctcccacaatctccaaaagataatgccaaaacctgggtatcttcctggatttgtgaatgacgtacaggtattcatttattcattggtacacattctgtatgctgctgttttcaagttggcaaattaagcatatgataaaatcccaaaact

SEQ ID NO: 33 - protein, 563 aa, Homo sapiens
MIKRRAHPGAGGDRTRPRRRRSTESWIERCLNESENKRYSSHTSLGNVSNDENEEKENNRASKPHSTPATLQWLEENYEIAEGVCIPRSALYMHYLDFCEKNDTQPVNAASFGKIIRQQFPQLTTRRLGTRGQSKYHYYGIAVKESSQYYDVMYSKKGAAWVSETGKKEVSKQTVAYSPRSKLGTLLPEFPNVKDLNLPASLPEEKVSTFIMMYRTHCQRILDTVIRANFDEVQSFLLHFWQGMPPHMLPVLGSSTVVNIVGVCDSILYKAISGVLMPTVLQALPDSLTQVIRKFAKQLDEWLKVALHDLPENLRNIKFELSRRFSQILRRQTSLNHLCQASRTVIHSADITFQMLEDWRNVDLNSITKQTLYTMEDSRDEHRKLITQLYQEFDHLLEEQSPIESYIEWLDTMVDRCVVKVAAKRQGSLKKVAQQFLLMWSCFGTRVIRDMTLHSAPSFGSFHLIHLMFDDYVLYLLESLHCQERANELMRAMKGEGSTAEVREEIILTEAAAPTPSPVPSFSPAKSATSVEVPPPSSPVSNPSPEYTGLSTTGNGKSFKNFG

SEQ ID NO: 34 - DNA, 558 bp, Homo sapiens
acagacaaatggtttatctactattggagccatgggtcctgggaatattggaccaccccaaatagaagagctcaaagtcatccctgaaaccagcgaggaaaataatgaggacatctggaattcagaagagattccagaaggagcagaatatgatgatatgtgggatgttagagaaatcccagagtatgagattatattcagacagcaggtgggaactgaagatatatttttagggttgtcaaaaaaggactcctcaacaggttgttgcagtgaactagtggctaaaattaaattgccaaatacaaacccttctgatattcaaattgatatccaggaaacaatccttgaccttcgtactcctcagaagaagctgttgataactcttcctgagctggtggaatgtaccagtgccaaagcattctatatcccagagactgaaactcttgaaatccctatgactatgaaaagagagttagatattgctaatttcttctgaaactgcatgaaaaagataaaaagtagtaaaatggcattggtaacaataaaaaaactttgaaa

SEQ ID NO: 35 - protein, 164 aa, Homo sapiens
QTNGLSTIGAMGPGNIGPPQIEELKVIPETSEENNEDIWNSEEIPEGAEYDDMWDVREIPEYEIIFRQQVGTEDIFLGLSKKDSSTGCCSELVAKIKLPNTNPSDIQIDIQETILDLRTPQKKLLITLPELVECTSAKAFYIPETETLEIPMTMKRELDIANFF

SEQ ID NO: 36 - DNA, 538 bp, Homo sapiens
ggcacgaggtcgaaaactcctggaaccctctgagagaggacagtttccagactcctcggtagggacgcgggaagagaccatgtgtgggaaatatgagtgagcatgtgagaacaagatcccaatcctcagaaagaggaaatgaccaagagtcttcccagccagttggatctgtgattgtccaggagcccactgaggaaaaacgtcaagaagaggaaccaccaactgataatcagggtattgcacctagtggggagatcgaaaatgaaggagcacctgccgttcaagggcctgacatggaagcttttcaacaggaactggctctgcttaagatagaggatgagcctggagatggtcctgatgtcagggaggggattatgcccacttttgatctcactaaagtgctggaagcaggtgatgcgcaaccataggtttcaagcaagacaaatgaagactgaaaccaagaacgttattcttaatctggaaatttgactgataatattctcttaataaagttttaagttttctgcaaagaaaaaaa

SEQ ID NO: 37 - protein, 111 aa, Homo sapiens
MSEHVRTRSQSSERGNDQESSQPVGSVIVQEPTEEKRQEEEPPTDNQGIAPSGEIENEGAPAVQGPDMEAFQQELALLKIEDEPGDGPDVREGIMPTFDLTKVLEAGDAQP

(19) United States
(12) Patent Application Publication
(10) Pub. No.: US 2003/0044813 A1
(43) Pub. Date: Mar. 6, 2003

(54) Cancer-testis antigens

(76) Inventors: Lloyd J. Old, New York, NY (US); Matthew J. Scanlan, New York, NY (US); Yao-Tseng Chen, New York, NY (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/054,683

(22) Filed: Jan. 22, 2002

Related U.S. Application Data
(60) Provisional application No. 60/280,718, filed on Mar. 30, 2001.
(60) Provisional application No. 60/285,154, filed on Apr. 20, 2001.
(60) Provisional application No. 60/327,432, filed on Oct. 5, 2001.

(51) Int. Cl. C12Q 1/68; G01N 33/574

(52) U.S. Cl. 435/6; 435/7.23

(57) ABSTRACT

Cancer-testis (CT) antigens have been identified by screening public databases for transcripts that are expressed in tumor tissues and a limited set of normal tissues, or by screening for genes that are expressed in cancer and testis tissues (but not other normal tissues). The invention relates to nucleic acids and encoded polypeptides which are CT antigens expressed in patients afflicted with cancer. The invention provides, inter alia, isolated nucleic acid molecules, expression vectors containing those molecules and host cells transfected with those molecules. The invention also provides isolated proteins and peptides, antibodies to those proteins and peptides and cytotoxic T lymphocytes which recognize the proteins and peptides. Fragments of the foregoing including functional fragments and variants also are provided. Kits containing the foregoing molecules additionally are provided. The molecules provided by the invention can be used in the diagnosis, monitoring, research, or treatment of conditions characterized by the expression of one or more CT antigens.

### RELATED APPLICATIONS

This application claims priority under 35 U.S.C. §119(e) to U.S. provisional application No. 60/280,718 filed Mar. 30, 2001, U.S. provisional application No. 60/285,154 filed Apr. 20, 2001, and U.S. provisional application No. 60/327,432 filed Oct. 5, 2001.

### FIELD OF THE INVENTION

The invention relates to nucleic acids and encoded polypeptides which are novel cancer-testis antigens expressed in a variety of cancers. The invention also relates to agents which bind the nucleic acids or polypeptides. The nucleic acid molecules, polypeptides coded for by such molecules and peptides derived therefrom, as well as related antibodies and cytolytic T lymphocytes, are useful, inter alia, in diagnostic and therapeutic contexts.

### BACKGROUND OF THE INVENTION

It is a little acknowledged fact that the discipline of tumor immunology has been the source of many findings of critical importance in cancer-related as well as cancer-unrelated fields. For example, it was the search for tumor antigens that led to the discovery of the CD8 T cell antigen (1) and the concept of differentiation antigens (2) (and the CD system for classifying cell surface antigens), and to the discovery of p53 (3). The immunogenetic analysis of resistance to viral leukemogenesis provided the first link between the MHC and disease susceptibility (4), and interest in the basis for non-specific immunity to cancer gave rise to the discovery of TNF (5).

Another area of tumor immunology that holds great promise is the category of antigens referred to as cancer/testis (CT) antigens, first recognized as targets for CD8 T cell recognition of autologous human melanoma cells (6, 7). The molecular definition of these antigens was a culmination of prior efforts to establish systems and methodologies for the unambiguous analysis of humoral (8) and cellular (9) immune reactions of patients to autologous tumor cells (autologous typing), and this approach of autologous typing also led to the development of SEREX (serological analysis of cDNA expression libraries) for defining the molecular structure of tumor antigens eliciting a humoral immune response (10).

Although the usefulness of the known CT antigens in the diagnosis and therapy of cancer is accepted, the expression of these antigens in tumors of various types and sources is not universal. Accordingly, there is a need to identify additional CT antigens to provide more targets for diagnosis and therapy of cancer, and for the development of pharmaceuticals useful in diagnostic and therapeutic applications.

### SUMMARY OF THE INVENTION

Bioinformatic analysis of sequence databases has been applied to identify sequences having expression characteristics that fit the profile of cancer/testis antigens. Several novel cancer/testis antigens and cancer associated antigens have been identified. The invention provides, inter alia, isolated nucleic acid molecules, expression vectors containing those molecules and host cells transfected with those molecules. The invention also provides isolated proteins and peptides, antibodies to those proteins and peptides and CTLs which recognize the proteins and peptides. Fragments and variants of the foregoing also are provided. Kits containing the foregoing molecules additionally are provided. The foregoing can be used in the diagnosis, monitoring, research, or treatment of conditions characterized by the expression of one or more cancer-testis and/or cancer associated antigens.

Prior to the present invention, only a handful of cancer/testis antigens had been identified in the past 20 years. The invention involves the surprising discovery of several sequence clusters (UniGene) in sequence databases that have expression patterns that fit the profile of cancer-testis antigens. Other sequence clusters fit the profile of cancer associated antigens. The knowledge that these sequence clusters have these certain expression patterns makes the sequences useful in the diagnosis, monitoring and therapy of a variety of cancers.

The invention involves the use of a single material, a plurality of different materials and even large panels and combinations of materials. For example, a single gene, a single protein encoded by a gene, a single functional fragment thereof, a single antibody thereto, etc. can be used in methods and products of the invention. Likewise, pairs, groups and even panels of these materials and optionally other CT antigen genes and/or gene products can be used for diagnosis, monitoring and therapy. The pairs, groups or panels can involve 2, 3, 4, 5 or more genes, gene products, fragments thereof or agents that recognize such materials. A plurality of such materials are not only useful in monitoring, typing, characterizing and diagnosing cells abnormally expressing such genes, but a plurality of such materials can be used therapeutically. An example of the use of a plurality of such materials for the prevention, delay of onset, amelioration, etc. of cancer cells, which express or will express such genes prophylactically or acutely. Any and all combinations of the genes, gene products, and materials which recognize the genes and gene products can be tested and identified for use according to the invention. It would be far too lengthy to recite all such combinations; those skilled in the art, particularly in view of the teaching contained herein, will readily be able to determine which combinations are most appropriate for which circumstances.

As will be clear from the following discussion, the invention has in vivo and in vitro uses, including for therapeutic, diagnostic, monitoring and research purposes. One aspect of the invention is the ability to fingerprint a cell expressing a number of the genes identified according to the invention by, for example, quantifying the expression of such gene products. Such fingerprints will be characteristic, for example, of the stage of the cancer, the type of the cancer, or even the effect in subjects or animal models of a therapy on a cancer. Cells also can be screened to determine whether such cells abnormally express the genes identified according to the invention.

According to one aspect of the invention, methods of diagnosing a disorder are provided. The methods include contacting a non-testis biological sample isolated from a subject with an agent that specifically binds to a nucleic acid molecule, an expression product thereof, or a fragment of the expression product thereof complexed with an HLA molecule, wherein the nucleic acid molecule comprises a nucleotide sequence selected from the group consisting of SEQ ID NOs:18, 20 and 22, and determining the interaction between the agent and the nucleic acid molecule or the expression product to diagnose the cancer in the subject.

In certain embodiments, the agent is selected from the group consisting of (a) a nucleic acid molecule comprising a nucleotide sequence selected from the group consisting of SEQ ID NOs:18, 20 and 22 or a fragment thereof, (b) an antibody that binds to an expression product of a nucleic acid molecule comprising a nucleotide sequence selected from the group consisting of SEQ ID NOs:18, 20 and 22, and (c) an agent that binds to a complex of an HLA molecule and a fragment of an expression product of a nucleic acid molecule comprising a nucleotide sequence selected from the group consisting of SEQ ID NOs:18, 20 and 22.

In other embodiments, the cancer is characterized by expression of a plurality of human CT antigen precursors and wherein the agent is a plurality of agents, each of which is specific for a different human CT antigen precursor, and wherein said plurality of agents is at least 2, at least 3, at least 4, at least 5, at least 6, at least 7, or at least 8, at least 9 or at least 10 such agents.

According to another aspect of the invention, methods of diagnosing a cancer are provided. The methods include contacting a non-testis, non-brain biological sample isolated from a subject with an agent that specifically binds to a nucleic acid molecule, an expression product thereof, or a fragment of an expression product thereof complexed with an HLA molecule, wherein the nucleic acid molecule comprises a nucleotide sequence set forth as SEQ ID NO:32, and determining the interaction between the agent and the nucleic acid molecule or the expression product to diagnose the cancer in the subject.

According to a further aspect of the invention, other methods of diagnosing a cancer are provided. The methods include contacting a non-testis, non-ovary, non-cervix, non-lung biological sample isolated from a subject with an agent that specifically binds to a nucleic acid molecule, an expression product thereof, or a fragment of an expression product thereof complexed with an HLA molecule, wherein the nucleic acid molecule comprises a nucleotide sequence set forth as SEQ ID NO:34, and determining the interaction between the agent and the nucleic acid molecule or the expression product to diagnose the cancer in the subject.

According to a still another aspect of the invention, other methods of diagnosing a cancer are provided. These methods include contacting a non-testis, non-ovary, non-lung, non-breast, non-prostate, non-colon biological sample isolated from a subject with an agent that specifically binds to a nucleic acid molecule, an expression product thereof, or a fragment of an expression product thereof complexed with an HLA molecule, wherein the nucleic acid molecule comprises a nucleotide sequence set forth as SEQ ID NO:36, and determining the interaction between the agent and the nucleic acid molecule or the expression product to diagnose the cancer in the subject.

In another aspect of the invention, methods for determining regression, progression or onset of a cancer characterized by expression of abnormal levels of a protein encoded by a nucleic acid molecule comprising a nucleotide sequence selected from the group consisting of SEQ ID NOs:18, 20 and 22 are provided. The methods include monitoring a plurality of non-testis samples obtained at different times from a subject who has or is suspected of having the cancer, for a parameter selected from the group consisting of (i) the protein, (ii) a peptide derived from the protein, (iii) an antibody which selectively binds the protein or peptide, (iv) cytolytic T cells specific for a complex of the peptide derived from the protein and an MHC molecule, and (v) a nucleic acid molecule comprising a nucleotide sequence selected from the group consisting of SEQ ID NOs:18, 20 and 22. The methods also include comparing the parameters from the plurality of samples to determine regression, progression or onset of the cancer.

In some embodiments, the sample is a body fluid, a body effusion, cell or a tissue. In other embodiments, the step of monitoring comprises contacting the sample with a detectable agent selected from the group consisting of (a) an antibody which selectively binds the protein of (i) or the peptide of (ii), (b) a protein or peptide which binds the antibody of (iii), (c) a cell which presents the complex of the peptide and MHC molecule of (iv), and (d) at least one nucleic acid probe or primer that hybridizes to the nucleic acid molecule of (v) or its complement. Preferably the antibody, the protein, the peptide, the cell or the nucleic acid probe or primer is labeled with a radioactive label or an enzyme. In further embodiments, the protein is a plurality of proteins, the parameter is a plurality of parameters, each of the plurality of parameters being specific for a different of the plurality of proteins, at least one of which is a CT antigen protein encoded by a nucleic acid molecule comprising a nucleotide sequence selected from the group consisting of SEQ ID NOs:18, 20 and 22.

According to yet another aspect of the invention, pharmaceutical preparations for a human subject are provided. The pharmaceutical preparations include an agent which, when administered to the subject, enriches selectively the presence of complexes of an HLA molecule and a human CT antigen peptide, and a pharmaceutically acceptable carrier. The human CT antigen peptide is a fragment of a human CT antigen encoded by a nucleic acid molecule comprising a nucleotide sequence selected from the group consisting of SEQ ID NOs:18, 20 and 22.

In some embodiments, the foregoing pharmaceutical preparations includes a plurality of agents, each of which enriches selectively in the subject complexes of an HLA molecule and a different human CT antigen peptide, wherein at least one of the human CT antigens is encoded by a nucleic acid molecule comprising a nucleotide sequence selected from the group consisting of SEQ ID NOs:18, 20 and 22.

In other embodiments of the foregoing pharmaceutical preparations, the agent is selected from the group consisting of (1) an isolated polypeptide comprising the human CT antigen peptide, (2) an isolated nucleic acid operably linked to a promoter for expressing the isolated polypeptide, (3) a host cell expressing the isolated polypeptide, and (4) isolated complexes of the polypeptide and an HLA molecule.

In still other embodiments, the agent is a cell expressing an isolated polypeptide comprising the human CT antigen peptide, or a cell expressing an isolated polypeptide comprising the human CT antigen peptide, and an HLA molecule that binds the polypeptide. n certain of these embodiments, the cell expresses the polypeptide and/or the HLA molecule recombinantly. Preferably, the foregoing cells are nonproliferative.

The foregoing pharmaceutical preparations preferably also include an adjuvant.

According to another aspect of the invention, compositions are provided. The compositions include an isolated agent that binds selectively a polypeptide comprising an amino acid sequence selected from the group consisting of SEQ ID NOs:21, 23, 25, 27, 29, 31, 35 and 37. Preferably, the agent is an antibody or an antigen-binding fragment thereof More preferably, the antibody is a monoclonal antibody, a chimeric antibody or a humanized antibody. Also provided are compositions of matter include one or more conjugates of the foregoing agents and a therapeutic or diagnostic agent. Preferably the therapeutic or diagnostic agent is a toxin.

Pharmaceutical compositions are provided in another aspect of the invention. The compositions include an isolated nucleic acid molecule comprising a nucleotide sequence selected from the group consisting of SEQ ID NOs:18, 20 and 22, and a pharmaceutically acceptable carrier. In some embodiments, the isolated nucleic acid molecule comprises at least two isolated nucleic acid molecules coding for two different polypeptides, each polypeptide comprising a different human CT antigen. In other embodiments, the foregoing pharmaceutical compositions also include an expression vector with a promoter operably linked to the isolated nucleic acid molecule. In still other embodiments, the foregoing pharmaceutical compositions also include a host cell recombinantly expressing the isolated nucleic acid molecule.

In a further aspect of the invention, additional pharmaceutical compositions are provided. The compositions include an isolated polypeptide comprising a polypeptide encoded by a nucleic acid molecule comprising a nucleotide sequence selected from the group consisting of SEQ ID NOs:18, 20 and 22, and a pharmaceutically acceptable carrier. In certain embodiments, the isolated polypeptide includes at least two different polypeptides, each comprising a different human CT antigen. In still other embodiments, the foregoing pharmaceutical compositions also include an adjuvant.

According to yet another aspect of the invention, protein microarrays are provided. The protein microarrays include at least one polypeptide encoded by a nucleic acid molecule comprising a nucleotide sequence selected from the group consisting of SEQ ID NOs:18, 20 and 22, or an antigenic fragment of the at least one polypeptide. In preferred embodiments, the at least one polypeptide comprises an amino acid sequence selected from the group consisting of SEQ ID NOs:19, 21 and 23.

According to yet a further aspect of the invention, protein microarrays are provided. The protein microarrays include at least one polypeptide encoded by a nucleic acid molecule comprising a nucleotide sequence selected from the group consisting of SEQ ID NOs:24, 26, 28 and 30, or an antigenic fragment of the polypeptide. Preferably, the at least one polypeptide comprises an amino acid sequence selected from the group consisting of SEQ ID NOs:25, 27, 29 and 31.

Additional protein microarrays are provided according to another aspect of the invention. The microarrays include at least one polypeptide encoded by a nucleic acid molecule comprising a nucleotide sequence selected from the group consisting of SEQ ID NOs:32, 34 and 36, or an antigenic fragment of the polypeptide. Preferably the at least one polypeptide comprises an amino acid sequence selected from the group consisting of SEQ ID NOs:33, 35 and 37.

In another aspect of the invention, protein microarrays are provided that include a plurality of antibodies or antigen-binding fragments thereof that specifically bind at least one polypeptide encoded by a nucleic acid molecule comprising a nucleotide sequence selected from the groups consisting of (i) SEQ ID NOs:18, 20 and 22; (ii) SEQ ID NOs:24, 26, 28 and 30; or (iii) SEQ ID NOs:32, 34 and 36; or antigenic fragments of the foregoing polypeptides. Preferably, the polypeptides include at least one amino acid sequence selected from the groups consisting of (i) SEQ ID NOs:19, 21 and 23; (ii) SEQ ID NOs:25, 27, 29 and 31; or (iii) SEQ ID NOs:33, 35 and 37.

According to still another aspect of the invention, nucleic acid microarrays are provided that include at least one nucleic acid molecule comprising a nucleotide sequence selected from the groups consisting of (i) SEQ ID NOs:18, 20 and 22; (ii) SEQ ID NOs:24, 26, 28 and 30; or (iii) SEQ ID NOs:32, 34 and 36; or fragments thereof of at least 20 nucleotides that selectively hybridizes to its complement in a biological sample.

Also provided in accordance with a further aspect of the invention are isolated fragments of a human CT antigen which, or a portion of which, binds a HLA molecule or a human antibody. The foregoing CT antigens are encoded by a nucleic acid molecule comprising a nucleotide sequence selected from the group consisting of SEQ ID NOs:18, 20 and 22. In some embodiments, the fragment is part of a complex with the HLA molecule. Preferably the fragment is between 8 and 12 amino acids in length.

According to yet another aspect of the invention, kits for detecting the expression of two or more human CT antigens are provided. The kits include two or more pairs of isolated nucleic acid molecules, each of which consists essentially of a nucleic acid molecule selected from the group consisting of (a) a 12-32 nucleotide contiguous segment of the nucleotide sequence of any of SEQ ID NOs:18, 20 or 22, and (b) complements of (a), wherein the contiguous segments are nonoverlapping, and wherein the nucleic acid molecules in each of the pairs are specific for a human CT antigen. In certain embodiments, the pair of isolated nucleic acid molecules is constructed and arranged to selectively amplify at least a fragment of an isolated nucleic acid molecule selected from the group consisting of SEQ ID NOs:18, 20 and 22.

Also provided in an additional aspect of the invention are methods for treating a subject with a cancer characterized by expression of a human CT antigen. The methods include administering to the subject an amount of an agent, which enriches selectively in the subject the presence of complexes of a HLA molecule and a human CT antigen peptide, effective to ameliorate the disorder. The human CT antigen peptide is a fragment of a human CT antigen encoded by a nucleic acid molecule comprising a nucleotide sequence selected from the group consisting of SEQ ID NOs:18, 20 and 22. In certain embodiments, the cancer is characterized by expression of a plurality of human CT antigens and wherein the agent is a plurality of agents, each of which enriches selectively in the subject the presence of complexes of an HLA molecule and a different human CT antigen peptide, wherein at least one of the human CT antigens is encoded by a nucleic acid molecule comprising a nucleotide sequence selected from the group consisting of SEQ ID NOs:18, 20 and 22. In other embodiments, the agent is an isolated polypeptide encoded by a nucleic acid molecule comprising a nucleotide sequence selected from the group consisting of SEQ ID NOs:18, 20 and 22.

In a further aspect of the invention, methods for treating a subject having a cancer characterized by expression of a human CT antigen in cells of the subject are provided. The methods include (i) removing an immunoreactive cell containing sample from the subject, (ii) contacting the immunoreactive cell containing sample to the host cell under conditions favoring production of cytolytic T cells against a human CT antigen peptide that is a fragment of the human CT antigen, and (iii) introducing the cytolytic T cells to the subject in an amount effective to lyse cells which express the human CT antigen. In these methods, the host cell is transformed or transfected with an expression vector comprising an isolated nucleic acid molecule operably linked to a promoter, wherein the isolated nucleic acid molecule comprises a nucleotide sequence selected from the group consisting of SEQ ID NOs:18, 20 and 22. In different embodiments of the foregoing methods, the host cell recombinantly or endogenously expresses an HLA molecule which binds the human CT antigen peptide.

In still another aspect of the invention, methods for treating a subject having a cancer characterized by expression of a human CT antigen in cells of the subject are provided. The methods include (i) identifying a nucleic acid molecule expressed by the cells of the cancer, wherein the nucleic acid molecule comprises a nucleotide sequence selected from the group consisting of SEQ ID NOs:18, 20 and 22, or a fragment thereof; (ii) transfecting a host cell with a nucleic acid molecule selected from the group consisting of (a) the nucleic acid molecule identified, (b) a fragment of the nucleic acid identified which includes a segment coding for a human CT antigen, (c) degenerates of (a) or (b) (iii) culturing said transfected host cells to express the transfected nucleic acid molecule, and; (iv) introducing an amount of said host cells or an extract thereof to the subject effective to increase an immune response against the cells of the subject associated with the condition.

In certain embodiments, the foregoing methods also include identifying an MHC molecule which presents a portion of an expression product of the nucleic acid molecule, wherein the host cell expresses the same MHC molecule as identified and wherein the host cell presents an MHC binding portion of the expression product of the nucleic acid molecule. In other embodiments, the immune response comprises a B-cell response or a T cell response. Preferably the response is a T-cell response which comprises generation of cytolytic T-cells specific for the host cells presenting the portion of the expression product of the nucleic acid molecule or cells of the subject expressing the human CT antigen.

In some embodiments, the nucleic acid molecule is selected from the group consisting of SEQ ID NOs:18, 20 and 22. In still other embodiments, the methods also include treating the host cells to render them non-proliferative.

According to yet another aspect of the invention, methods for treating or diagnosing or monitoring a subject having a cancer characterized by expression of a protein encoded by a nucleic acid molecule comprising a nucleotide sequence selected from the group consisting of SEQ ID NOs:18, 20, and 22, in cells or tissues other than testis are provided. The methods include administering to the subject an antibody which specifically binds to the protein or a peptide derived therefrom, the antibody being coupled to a therapeutically or diagnostically useful agent, in an amount effective to treat, diagnose or monitor the condition.

In some embodiments, the antibody is a monoclonal antibody or an antigen-binding fragment thereof. Preferably the monoclonal antibody is a chimeric antibody or a humanized antibody.

In another aspect of the invention, methods are provided for treating a cancer characterized by expression of a protein encoded by a nucleic acid molecule comprising a nucleotide sequence selected from the group consisting of SEQ ID NOs:18, 20 and 22, in cells or tissues other than testis. The methods include administering to a subject one or more of the foregoing pharmaceutical compositions in an amount effective to prevent, delay the onset of, or inhibit the condition in the subject. In some embodiments, the methods also include first identifying that the subject expresses abnormal amounts of the protein in a non-testis tissue.

According to another aspect of the invention, methods for treating a subject having a cancer characterized by expression of a protein encoded by a nucleic acid molecule comprising a nucleotide sequence selected from the group consisting of SEQ ID NOs:18, 20 and 22, in cells or tissues other than testis are provided. The methods include (i) identifying cells from the subject which express abnormal amounts of the protein; (ii) isolating a sample of the cells; (iii) cultivating the cells, and (iv) introducing the cells to the subject in an amount effective to provoke an immune response against the cells. In certain embodiments, the methods also include rendering the cells non-proliferative, prior to introducing them to the subject.

Methods for treating a pathological cell condition characterized by expression of a protein encoded by a nucleic acid molecule comprising a nucleotide sequence selected from the group consisting of SEQ ID NOs:18, 20 and 22, in cells or tissues other than testis, are provided in accordance with another aspect of the invention. The methods include administering to a subject in need thereof an effective amount of an agent which inhibits the expression or activity of the protein. In some embodiments, the agent is an inhibiting antibody which selectively binds to the protein and wherein the antibody is a monoclonal antibody, a chimeric antibody, a humanized antibody or an antibody fragment. In other embodiments, the agent is an antisense nucleic acid molecule which selectively binds to the nucleic acid molecule which encodes the protein.

Also provided in accordance with a further aspect of the invention are compositions of matter useful in stimulating an immune response to a plurality of a proteins comprising an amino acid sequence selected from the group consisting of SEQ ID NOs:19, 21 and 23. The compositions include a plurality of peptides that are fragments of the proteins, wherein the peptides bind to one or more MHC molecules presented on the surface of non-testis cells. In certain embodiments of the compositions, at least a portion of the plurality of peptides bind to MHC molecules and elicit a cytolytic response thereto. In other embodiments, at least one of the proteins is encoded by a nucleic acid molecule comprising a nucleotide sequence selected from the group consisting of SEQ ID NOs:18, 20 and 22. Preferably, the compositions also include an adjuvant. Preferred adjuvants include saponins, GM-CSF, interleukins, and immunostimulatory oligonucleotides.

In another aspect of the invention, isolated antibodies are provided which selectively binds to a complex of: (i) a peptide that is a fragment of a protein comprising an amino acid sequence selected from the group consisting of SEQ ID NOs:19, 21 and 23, and (ii) a MHC molecule to which binds the peptide to form the complex, wherein the isolated antibody does not bind to (i) or (ii) alone. Preferably the antibody is a monoclonal antibody, a chimeric antibody, a humanized antibody, or an antigen-binding fragment thereof.

According to a further aspect of the invention, methods are provided for treating or diagnosing or monitoring a subject having a cancer characterized by expression of a protein encoded by a nucleic acid molecule comprising a nucleotide sequence selected from the group consisting of SEQ ID NOs:18, 20, and 22, in cells or tissues other than testis. The methods include administering to the subject the foregoing antibodies, in an amount effective to treat, diagnose or monitor the condition. Preferably the antibodies are coupled to one or more therapeutically or diagnostically useful agents.

The invention also involves the use of the genes, gene products, fragments thereof, agents which bind thereto, and so on in the preparation of medicaments. A particular medicament is for treating cancer.

These and other aspects of the invention will be described in further detail in connection with the detailed description of the invention.

### BRIEF DESCRIPTION OF THE DRAWINGS

FIG. 1 shows a digitized image of RT-PCR expression analysis of cancer/testis-associated Unigene clusters in normal adult tissues and cancer.

FIG. 1A shows the expression of mRNA transcripts in normal adult tissue following 35 cycles of PCR. Lane 1, brain; 2, kidney; 3, liver; 4, pancreas; 5, placenta; 6, testis; 7, small intestine; 8, heart; 9, prostate; 10, adrenal gland; 11, spleen; 12, colon; 13, stomach; 14, lung; 15, bladder; 16, ovary; 17, mammary gland; 18, cervix; 19, skeletal muscle. The majority of transcripts are testis-specific, with the exceptions of Hs.183009, Hs.293317, Hs. 128836, and Hs.130926. Expression of Hs.130926 was used as a positive control for cDNA template integrity of the various tissue samples.

FIG. 1B shows RT-PCR analysis of CT15/Hs.177959 mRNA expression in renal cancer (RCC1, RCC6, RCC5), CT16/Hs245431 mRNA expression in melanoma (Mel-1 and Mel-11) and breast cancer; and CT17/Hs.178062 mRNA expression in breast cancer (BR-297), renal cancer (RCC5) and melanoma (Mel-1).

### DETAILED DESCRIPTION OF THE INVENTION

As a consequence of T cell epitope cloning and SEREX analysis, a growing number of cancer-testis (CT) antigens have now been defined. See Table 1 and references cited therein. There are now 14 genes or gene families identified that code for presumptive cancer-testis antigens.

TABLE 1
Cancer-testis (CT) antigens

| | | | Chromosome | Detection | |
|---|---|---|---|---|---|
| CT* | System | # Genes | Location | System** | Refs. |
| 1 | MAGE | 16 | Xq28/Xp21 | T, Ab | 7, 10, 12, |
| | | | | | 13 |
| 2 | BAGE | 2 | Unknown | T | 14 |
| 3 | GAGE | 9 | Xp11 | T | 15, 16 |
| 4 | SSX | >5 | Xp11 | Ab | 10, 17 |
| 5 | NY-ESO-1 | 2 | Xq28 | Ab, T, | 18, 19 |
| | LAGE-1 | | | RDA | |
| 6 | SCP-1 | 3 | 1p12-p13 | Ab | 20 |
| 7 | CT7/MAGE-C1 | 1 | Xq26 | Ab, RDA | 21, 22 |
| 8 | CT8 | 1 | Unknown | Ab | 23 |
| 9 | CT9 | 1 | 1p | Ab | 24 |
| 10 | CT10/MAGE-C2 | 1 | Xq27 | RDA, Ab | 25, 26 |
| 11 | CT11p | 1 | Xq26-Xq27 | *** | 27 |
| 12 | SAGE | 1 | Xq28 | RDA | 28 |
| 13 | cTAGE-1 | 1 | 18p11 | Ab | 29 |
| 14 | OY-TES-1 | 2 | 12p12-p13 | Ab | 30 |

*Numbered according to the CT nomenclature proposed by Old & Chen (11).
**Ab = Antibody, T = CD8+ T cell, RDA = representational difference analysis.
***Defined by differential mRNA expression in a parental vs. metastatic melanoma cell variant.

A thorough analysis of these gene reveals that they encode products with the following characteristics.

i) mRNA expression in normal tissues is restricted to testis, fetal ovary, and placenta, with little or no expression detected in adult ovary.

ii) mRNA expression in cancers of diverse origin is common—up to 30-40% of a number of different cancer types, e.g., melanoma, bladder cancer, sarcoma express one or more CT antigens.

iii) The X chromosome codes for the majority of CT antigens, but a number of more recently defined CT coding genes have a non-X chromosomal locus.

iv) In normal adult testis, expression of CT antigens is primarily restricted to immature germ cells—, e.g., spermatogonia (31). However, a recently defined CT antigen, OY-TES-1, is clearly involved in late stages of sperm maturation (see below). In fetal ovary, immature germ cells (oogonia/primary oocytes) express CT antigens, whereas oocytes in the resting primordial follicles do not (32). In fetal placenta, both cytotrophoblast and syncytiotrophoblast express CT antigens, but in term placenta, CT antigen expression is weak or absent (33).

v) A highly variable pattern of CT antigen expression is found in different cancers, from tumors showing only single positive cells or small cluster of positive cells to other tumors with a generally homogeneous expression pattern (31, 34).

vi) The function of most CT antigens is unknown, although some role in regulating gene expression appears likely. Two CT antigens, however, have known roles in gamete development—SCP-1, the synaptonemal complex protein, is involved in chromosomal reduction during meiosis (35), and OY-TES-1 is a proacrosin binding protein sp32 precursor thought to be involved in packaging acrosin in the acrosome in the sperm head (36).

vii) There is increasing evidence that CT expression is correlated with tumor progression and with tumors of higher malignant potential. For instance, a higher frequency of MAGE mRNA expression is found in metastatic vs. primary melanoma (37) and in invasive vs. superficial bladder cancer (38), and NY-ESO-1 expression in bladder cancer is correlated with high nuclear grade (39).

viii) There appears to be considerable variation in the inherent immunogenicity of different CT antigens as indicated by specific CD8⁺ T cell and antibody responses in patients with antigen positive tumors. To date, NY-ESO-1 appears to have the strongest spontaneous immunogenicity of any of the CT antigens—e.g., up to 50% of patients with advanced NY-ESO-1⁺ tumors develop humoral and cellular immunity to NY-ESO-1 (40, 41).

These characteristics indicate the desirability of cancer-testis antigens for use in diagnostics and therapeutics. These characteristics also provide a basis for the identification of additional cancer-testis antigens.

While others have attempted to identify cancer related sequences in public databases by the use of bioinformatics techniques, (e.g., database mining plus rapid screening by fluorescent-PCR expression, Loging et al., Genome Res 10(9):1393-402, 2000), these techniques have not focused on the identification of nucleic acid sequences that the preferred cancer-testis antigen profile. In particular, the present invention includes the identification of cancer-testis sequences by more stringent criteria. The database analysis criteria fro identifying cancer-testis antigen sequences include the requirement that the sequences are expressed in cancers from at least two different tissues, and preferably are expressed in cancers from at least three different tissues. In addition, the sequences preferably have normal tissue expression restricted to one or more tissue selected from the group consisting of testis, placenta and ovary (preferably only fetal ovary).

In the above summary and in the ensuing description, lists of sequences are provided. The lists are meant to embrace each single sequence separately, two or more sequences together where they form a part of the same gene, any combination of two or more sequences which relate to different genes, including and up to the total number on the list, as if each and every combination were separately and specifically enumerated. Likewise, when mentioning fragment size, it is intended that a range embrace the smallest fragment mentioned to the full-length of the sequence (less one nucleotide or amino acid so that it is a fragment), each and every fragment length intended as if specifically enumerated. Thus, if a fragment could be between 10 and 15 in length, it is explicitly meant to mean 10, 11, 12, 13, 14, or 15 in length.

The summary and the claims mention antigen precursors and antigens. As used in the summary and in the claims, a precursor is substantially the full-length protein encoded by the coding region of the isolated nucleic acid and the antigen is a peptide which complexes with MHC, preferably HLA, and which participates in the immune response as part of that complex. Such antigens are typically 9 amino acids long, although this may vary slightly.

As used herein, a subject is a human, non-human primate, cow, horse, pig, sheep, goat, dog, cat or rodent. In all embodiments human cancer antigens and human subjects are preferred.

The present invention in one aspect involves the identification of human CT antigens using autologous antisera of subjects having cancer. The sequences representing CT antigen genes identified according to the methods described herein are presented in the attached Sequence Listing. The nature of the sequences as encoding CT antigens recognized by the immune systems of cancer patients is, of course, unexpected.

The invention thus involves in one aspect CT antigen polypeptides, genes encoding those polypeptides, functional modifications and variants of the foregoing, useful fragments of the foregoing, as well as diagnostics and therapeutics relating thereto.

Homologs and alleles of the CT antigen nucleic acids of the invention can be identified by conventional techniques. Thus, an aspect of the invention is those nucleic acid sequences which code for CT antigen precursors.

The term “high stringency hybridization conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high stringency conditions, as used herein, refers, for example, to hybridization at 65° C. in hybridization buffer (3.5× SSC, 0.02% Ficoll, 0.02% polyvinyl pyrrolidone, 0.02% Bovine Serum Albumin, 2.5 mM NaH₂PO₄(pH 7), 0.5% SDS, 2 mM EDTA). SSC is 0.15M sodium chloride/0.15M sodium citrate, pH 7; SDS is sodium dodecyl sulphate; and EDTA is ethylenediaminetetracetic acid. After hybridization, the membrane upon which the DNA is transferred is washed, for example, in 2× SSC at room temperature and then at 0.1-0.5× SSC/0.1× SDS at temperatures up to 68° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of CT antigen nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

In general homologs and alleles typically will share at least 75% nucleotide identity and/or at least 90% amino acid identity to the sequences of CT antigen nucleic acid and polypeptides, respectively, in some instances will share at least 90% nucleotide identity and/or at least 95% amino acid identity and in still other instances will share at least 95% nucleotide identity and/or at least 99% amino acid identity. The homology can be calculated using various, publicly available software tools developed by NCBI (Bethesda, Md.) that can be obtained through the internet (ftp:/ncbi.nlm.nih.gov/pub/). Exemplary tools include the BLAST software available at http://www.ncbi.nlm.nih.gov, using default settings. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In screening for CT antigen genes, a Southern blot may be performed using the foregoing conditions, together with a radioactive probe. After washing the membrane to which the DNA is finally transferred, the membrane can be placed against X-ray film to detect the radioactive signal. In screening for the expression of CT antigen nucleic acids, Northern blot hybridizations using the foregoing can be performed on samples taken from cancer patients or subjects suspected of having a condition characterized by expression of CT antigen genes. Amplification protocols such as polymerase chain reaction using primers which hybridize to the sequences presented also can be used for detection of the CT antigen genes or expression thereof.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating CT antigen polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as antigenicity, enzymatic activity, receptor binding, formation of complexes by binding of peptides by MHC class I and class II molecules, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention also provides isolated fragments of CT antigen nucleic acid sequences or complements thereof, and in particular unique fragments. A unique fragment is one that is a ‘signature’ for the larger nucleic acid. It, for example, is long enough to assure that its precise sequence is not found in molecules within the human genome outside of the CT antigen nucleic acids defined above (and human alleles). Those of ordinary skill in the art may apply routine procedures to determine if a fragment is unique within the human genome, such as the use of publicly available sequence comparison software to selectively distinguish the sequence fragment of interest from other sequences in the human genome, although in vitro confirmatory hybridization and sequencing analysis may be performed.

Fragments can be used as probes in Southern and Northern blot assays to identify CT antigen nucleic acids, or can be used in amplification assays such as those employing PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the CT antigen polypeptides, useful, for example, in the preparation of antibodies, and in immunoassays. Fragments further can be used as antisense molecules to inhibit the expression of CT antigen nucleic acids and polypeptides, particularly for therapeutic purposes as described in greater detail below.

As mentioned above, this disclosure intends to embrace each and every fragment of each sequence, beginning at the first nucleotide, the second nucleotide and so on, up to 8 nucleotides short of the end, and ending anywhere from nucleotide number 8, 9, 10 and so on for each sequence, up to the entire length of the disclosed sequence. Preferred fragments are those useful as amplification primers, e.g., typically between 12 and 32 nucleotides (e.g. 12, 13, 14, 15, 16, 17, 18, 19, 20, 21, 22, 23, 24, 25, 26, 27, 28, 29, 30, 31 and 32) in length.

Those skilled in the art are well versed in methods for selecting such sequences, typically on the basis of the ability of the fragment to selectively distinguish the sequence of interest from other sequences in the human genome of the fragment to those on known databases typically is all that is necessary, although in vitro confirmatory hybridization and sequencing analysis may be performed.

Especially preferred fragment include nucleic acids encoding a series of epitopes, known as “polytopes”. The epitopes can be arranged in sequential or overlapping fashion (see, e.g., Thomson et al., Proc. Natl. Acad. Sci. USA 92:5845-5849, 1995; Gilbert et al., Nature Biotechnol. 15:1280-1284, 1997), with or without the natural flanking sequences, and can be separated by unrelated linker sequences if desired. The polytope is processed to generated individual epitopes which are recognized by the immune system for generation of immune responses.

Thus, for example, peptides derived from a polypeptide having an amino acid sequence encoded by one of the nucleic acid disclosed herein, and which are presented by MHC molecules and recognized by CTL or T helper lymphocytes, can be combined with peptides from one or more other CT antigens (e.g. by preparation of hybrid nucleic acids or polypeptides) to form “polytopes”. The two or more peptides (or nucleic acids encoding the peptides) can be selected from those described herein, or they can include one or more peptides of previously known CT antigens. Exemplary cancer associated peptide antigens that can be administered to induce or enhance an immune response are derived from tumor associated genes and encoded proteins including MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, GAGE-9, BAGE-1, RAGE-1, LB33/MUM-1, PRAME, NAG, MAGE-B2, MAGE-B3, MAGE-B4, tyrosinase, brain glycogen phosphorylase, Melan-A, MAGE-C 1, MAGE-C2, MAGE-C3, MAGE-C4, MAGE-C5, NY-ESO-1, LAGE-1, SSX-1, SSX-2 (HOM-MEL-40), SSX-4, SSX-5, SCP-1 and CT-7. See, for example, PCT application publication no. WO96/10577. Other examples will be known to one of ordinary skill in the art and can be used in the invention in a like manner as those disclosed herein. Other examples of HLA class I and HLA class II binding peptides will be known to one of ordinary skill in the art. For example, see the following references: Coulie, Stem Cells 13:393-403, 1995; Traversari et al., J. Exp. Med. 176:1453-1457, 1992; Chaux et al., J. Immunol. 163:2928-2936, 1999; Fujie et al., Int. J. Cancer 80:169-172, 1999; Tanzarella et al., Cancer Res. 59:2668-2674, 1999; van der Bruggen et al., Eur. J. Immunol. 24:2134-2140, 1994; Chaux et al., J. Exp. Med. 189:767-778, 1999; Kawashima et al, Hum. Immunol. 59:1-14, 1998; Tahara et al., Clin. Cancer Res. 5:2236-2241, 1999; Gaugler et al., J. Exp. Med. 179:921-930, 1994; van der Bruggen et al., Eur. J. Immunol. 24:3038-3043, 1994; Tanaka et al., Cancer Res. 57:4465-4468, 1997; Oiso et al., Int. J. Cancer 81:387-394, 1999; Herman et al., Immunogenetics 43:377-383, 1996; Manici et al., J. Exp. Med. 189:871-876, 1999; Duffour et al., Eur. J. Immunol. 29:3329-3337, 1999; Zorn et al., Eur. J. Immunol. 29:602-607, 1999; Huang et al., J. Immunol. 162:6849-6854, 1999; Boël et al., Immunity 2:167-175, 1995; Van den Eynde et al., J. Exp. Med. 182:689-698, 1995; De Backer et al., Cancer Res. 59:3157-3165, 1999; Jäger et al., J. Exp. Med. 187:265-270, 1998; Wang et al., J. Immunol. 161:3596-3606, 1998; Aarnoudse et al., Int. J. Cancer 82:442-448, 1999; Guilloux et al., J. Exp. Med. 183:1173-1183, 1996; Lupetti et al., J. Exp. Med. 188:1005-1016, 1998; Wölfel et al., Eur. J. Immunol. 24:759-764, 1994; Skipper et al., J. Exp. Med. 183:527-534, 1996; Kang et al., J. Immunol. 155:1343-1348, 1995; Morel et al., Int. J. Cancer 83:755-759, 1999; Brichard et al., Eur. J. Immunol. 26:224-230, 1996; Kittlesen et al., J. Immunol. 160:2099-2106, 1998; Kawakami et al., J. Immunol. 161:6985-6992, 1998; Topalian et al., J. Exp. Med. 183:1965-1971, 1996; Kobayashi et al., Cancer Research 58:296-301, 1998; Kawakami et al., J. Immunol. 154:3961-3968, 1995; Tsai et al., J. Immunol. 158:1796-1802, 1997; Cox et al., Science 264:716-719, 1994; Kawakami et al., Proc. Natl. Acad. Sci. USA 91:6458-6462, 1994; Skipper et al., J. Immunol. 157:5027-5033, 1996; Robbins et al., J. Immunol. 159:303-308, 1997; Castelli et al, J. Immunol. 162:1739-1748, 1999; Kawakami et al., J. Exp. Med. 180:347-352, 1994; Castelli et al., J. Exp. Med. 181:363-368, 1995; Schneider et al., Int. J. Cancer 75:451-458, 1998; Wang et al., J. Exp. Med. 183:1131-1140, 1996; Wang et al., J. Exp. Med. 184:2207-2216, 1996; Parkhurst et al., Cancer Research 58:4895-4901, 1998; Tsang et al., J. Natl Cancer Inst 87:982-990, 1995; Correale et al., J Natl Cancer Inst 89:293-300, 1997; Coulie et al., Proc. Natl. Acad. Sci. USA 92:7976-7980, 1995; Wölfel et al., Science 269:1281-1284, 1995; Robbins et al., J. Exp. Med. 183:1185-1192, 1996; Brandle et al., J. Exp. Med. 183:2501-2508, 1996; ten Bosch et al., Blood 88:3522-3527, 1996; Mandruzzato et al., J. Exp. Med. 186:785-793, 1997; Guéguen et al., J. Immunol. 160:6188-6194, 1998; Gjertsen et al., Int. J. Cancer 72:784-790, 1997; Gaudin et al., J. Immunol. 162:1730-1738, 1999; Chiari et al., Cancer Res. 59:5785-5792, 1999; Hogan et al., Cancer Res. 58:5144-5150, 1998; Pieper et al., J. Exp. Med. 189:757-765, 1999; Wang et al., Science 284:1351-1354, 1999; Fisk et al., J. Exp. Med. 181:2109-2117, 1995; Brossart et al., Cancer Res. 58:732-736, 1998; Röpke et al., Proc. Natl. Acad. Sci. USA 93:14704-14707, 1996; Ikeda et al., Immunity 6:199-208, 1997; Ronsin et al., J. Immunol. 163:483-490, 1999; Vonderheide et al., Immunity 10:673-679,1999.

One of ordinary skill in the art can prepare polypeptides comprising one or more CT antigen peptides and one or more of the foregoing cancer associated peptides, or nucleic acids encoding such polypeptides, according to standard procedures of molecular biology.

Thus polytopes are groups of two or more potentially immunogenic or immune response stimulating peptides which can be joined together in various arrangements (e.g. concatenated, overlapping). The polytope (or nucleic acid encoding the polytope) can be administered in a standard immunization protocol, e.g. to animals, to test the effectiveness of the polytope in stimulating, enhancing and/or provoking an immune response.

The peptides can be joined together directly or via the use of flanking sequences to form polytopes, and the use of polytopes as vaccines is well known in the art (see, e.g., Thomson et al., Proc. Acad. Natl. Acad. Sci USA 92(13):5845-5849, 1995; Gilbert et al., Nature Biotechnol. 15(12):1280-1284, 1997; Thomson et al., J. Immunol. 157(2):822-826, 1996; Tam et al., J. Exp. Med. 171(1):299-306, 1990). For example, Tam showed that polytopes consisting of both MHC class I and class II binding epitopes successfully generated antibody and protective immunity in a mouse model. Tam also demonstrated that polytopes comprising “strings” of epitopes are processed to yield individual epitopes which are presented by MHC molecules and recognized by CTLs. Thus polytopes containing various numbers and combinations of epitopes can be prepared and tested for recognition by CTLs and for efficacy in increasing an immune response.

It is known that tumors express a set of tumor antigens, of which only certain subsets may be expressed in the tumor of any given patient. Polytopes can be prepared which correspond to the different combination of epitopes representing the subset of tumor rejection antigens expressed in a particular patient. Polytopes also can be prepared to reflect a broader spectrum of tumor rejection antigens known to be expressed by a tumor type. Polytopes can be introduced to a patient in need of such treatment as polypeptide structures, or via the use of nucleic acid delivery systems known in the art (see, e.g., Allsopp et al., Eur. J. Immunol. 26(8):1951-1959, 1996). Adenovirus, pox viruses, Ty-virus like particles, adeno-associated virus, alphaviruses, plasmids, bacteria, etc. can be used in such delivery. One can test the polytope delivery systems in mouse models to determine efficacy of the delivery system. The systems also can be tested in human clinical trials.

In instances in which a human HLA class I molecule presents tumor rejection antigens derived from CT antigens, the expression vector may also include a nucleic acid sequence coding for the HLA molecule that presents any particular tumor rejection antigen derived from these nucleic acids and polypeptides. Alternatively, the nucleic acid sequence coding for such a HLA molecule can be contained within a separate expression vector. In a situation where the vector contains both coding sequences, the single vector can be used to transfect a cell which does not normally express either one. Where the coding sequences for a CT antigen precursor and the HLA molecule which presents it are contained on separate expression vectors, the expression vectors can be cotransfected. The CT antigen precursor coding sequence may be used alone, when, e.g. the host cell already expresses a HLA molecule which presents a CT antigen derived from precursor molecules. Of course, there is no limit on the particular host cell which can be used. As the vectors which contain the two coding sequences may be used in any antigen-presenting cells if desired, and the gene for CT antigen precursor can be used in host cells which do not express a HLA molecule which presents a CT antigen. Further, cell-free transcription systems may be used in lieu of cells.

As mentioned above, the invention embraces antisense oligonucleotides that selectively bind to a nucleic acid molecule encoding a CT antigen polypeptide, to reduce the expression of CT antigens. This is desirable in virtually any medical condition wherein a reduction of expression of CT antigens is desirable, e.g., in the treatment of cancer. This is also useful for in vitro or in vivo testing of the effects of a reduction of expression of one or more CT antigens.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon the sequences of nucleic acids encoding CT antigens, or upon allelic or homologous genomic and/or cDNA sequences, one of skill in the art can easily choose and synthesize any of a number of appropriate antisense molecules for use in accordance with the present invention.

In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases.

Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. Suitable antisense molecules can be identified by a “gene walk” experiment in which overlapping oligonucleotides corresponding to the CT antigen nucleic acid are synthesized and tested for the ability to inhibit expression, cause the degradation of sense transcripts, etc. Finally, although the listed sequences are cDNA sequences, one of ordinary skill in the art may easily derive the genomic DNA corresponding to the cDNA of a CT antigen. Thus, the present invention also provides for antisense oligonucleotides which are complementary to the genomic DNA corresponding to nucleic acids encoding CT antigens. Similarly, antisense to allelic or homologous cDNAs and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. Base analogs such as C-5 propyne modified bases also can be included (Nature Biotechnol. 14:840-844, 1996). The present invention, thus, contemplates pharmaceutical preparations containing modified antisense molecules that are complementary to and hybridizable with, under physiological conditions, nucleic acids encoding the CT antigen polypeptides, together with pharmaceutically acceptable carriers.

Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art, as further described below.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a CT antigen polypeptide or fragment or variant thereof. That heterologous DNA (RNA) is placed under operable control of transcriptional elements to permit the expression of the heterologous DNA in the host cell.

Preferred systems for mRNA expression in mammalian cells are those such as pRc/CMV or pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr Virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant for the expression of an antigen is disclosed by Warnier et al., in intradermal injection in mice for immunization against P1A (Int. J. Cancer, 67:303-310, 1996).

The invention also embraces so-called expression kits, which allow the artisan to prepare a desired expression vector or vectors. Such expression kits include at least separate portions of a vector and one or more of the previously discussed CT antigen nucleic acid molecules. Other components may be added, as desired, as long as the previously mentioned nucleic acid molecules, which are required, are included. The invention also includes kits for amplification of a CT antigen nucleic acid, including at least one pair of amplification primers which hybridize to a CT antigen nucleic acid. The primers preferably are 12-32 nucleotides in length and are non-overlapping to prevent formation of “primer-dimers”. One of the primers will hybridize to one strand of the CT antigen nucleic acid and the second primer will hybridize to the complementary strand of the CT antigen nucleic acid, in an arrangement which permits amplification of the CT antigen nucleic acid. Selection of appropriate primer pairs is standard in the art. For example, the selection can be made with assistance of a computer program designed for such a purpose, optionally followed by testing the primers for amplification specificity and efficiency.

The invention also permits the construction of CT antigen gene “knock-outs” and “knock-ins” in cells and in animals, providing materials for studying certain aspects of cancer and immune system responses to cancer.

The invention also provides isolated polypeptides (including whole proteins and partial proteins) encoded by the foregoing CT antigen nucleic acids. Such polypeptides are useful, for example, alone or as fusion proteins to generate antibodies, as components of an immunoassay or diagnostic assay or as therapeutics. CT antigen polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including antigenic peptides (such as are presented by MHC molecules on the surface of a cell for immune recognition) also can be synthesized chemically using well-established methods of peptide synthesis.

A unique fragment of a CT antigen polypeptide, in general, has the features and characteristics of unique fragments as discussed above in connection with nucleic acids. As will be recognized by those skilled in the art, the size of the unique fragment will depend upon factors such as whether the fragment constitutes a portion of a conserved protein domain. Thus, some regions of CT antigens will require longer segments to be unique while others will require only short segments, typically between 5 and 12 amino acids (e.g. 5, 6, 7, 8, 9, 10, 11 or 12 or more amino acids including each integer up to the full length).

Fragments of a CT antigen polypeptide preferably are those fragments which retain a distinct functional capability of the polypeptide. Functional capabilities which can be retained in a fragment of a polypeptide include interaction with antibodies, interaction with other polypeptides or fragments thereof, selective binding of nucleic acids or proteins, and enzymatic activity. One important activity is the ability to act as a signature for identifying the polypeptide. Another is the ability to complex with HLA and to provoke in a human an immune response. Those skilled in the art are well versed in methods for selecting unique amino acid sequences, typically on the basis of the ability of the fragment to selectively distinguish the sequence of interest from non-family members. A comparison of the sequence of the fragment to those on known databases typically is all that is necessary.

The invention embraces variants of the CT antigen polypeptides described above. As used herein, a “variant” of a CT antigen polypeptide is a polypeptide which contains one or more modifications to the primary amino acid sequence of a CT antigen polypeptide. Modifications which create a CT antigen variant can be made to a CT antigen polypeptide 1) to reduce or eliminate an activity of a CT antigen polypeptide; 2) to enhance a property of a CT antigen polypeptide, such as protein stability in an expression system or the stability of protein-protein binding; 3) to provide a novel activity or property to a CT antigen polypeptide, such as addition of an antigenic epitope or addition of a detectable moiety; or 4) to provide equivalent or better binding to an HLA molecule. Modifications to a CT antigen polypeptide are typically made to the nucleic acid which encodes the CT antigen polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the CT antigen amino acid sequence. One of skill in the art will be familiar with methods for predicting the effect on protein conformation of a change in protein sequence, and can thus “design” a variant CT antigen polypeptide according to known methods. One example of such a method is described by Dahiyat and Mayo in Science 278:82-87, 1997, whereby proteins can be designed de novo. The method can be applied to a known protein to vary a only a portion of the polypeptide sequence. By applying the computational methods of Dahiyat and Mayo, specific variants of a CT antigen polypeptide can be proposed and tested to determine whether the variant retains a desired conformation.

In general, variants include CT antigen polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a CT antigen polypeptide by eliminating proteolysis by proteases in an expression system (e.g., dibasic amino acid residues in yeast expression systems in which KEX2 protease activity is present).

Mutations of a nucleic acid which encode a CT antigen polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant CT antigen polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a CT antigen gene or cDNA clone to enhance expression of the polypeptide. The activity of variants of CT antigen polypeptides can be tested by cloning the gene encoding the variant CT antigen polypeptide into a bacterial or mammalian expression vector, introducing the vector into an appropriate host cell, expressing the variant CT antigen polypeptide, and testing for a functional capability of the CT antigen polypeptides as disclosed herein. For example, the variant CT antigen polypeptide can be tested for binding to antibodies or T cells. Preferred variants are those that compete for binding with the original polypeptide for binding to antibodies or T cells. Preparation of other variant polypeptides may favor testing of other activities, as will be known to one of ordinary skill in the art.

The skilled artisan will also realize that conservative amino acid substitutions may be made in CT antigen polypeptides to provide functionally equivalent variants of the foregoing polypeptides, i.e., the variants retain the functional capabilities of the CT antigen polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the CT antigen polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide derived from a CT antigen polypeptide is presented by an MHC molecule and recognized by CTLs (e.g., as described in the Examples), one can make conservative amino acid substitutions to the amino acid sequence of the peptide, particularly at residues which are thought not to be direct contact points with the MHC molecule, i.e., the anchor residues that confer MHC binding. One of ordinary skill in the art will know these residues and will preferentially substitute other amino acid residues in the peptides in making variants. It is possible also to use other members of the consensus amino acids for a particular anchor residue. For example, consensus anchor residues for HLA-B35 are P in position 2 and Y, F, M, L or I in position 9. Therefore, if position 9 of a peptide was tyrosine (Y), one could substitute phenylalanine (F), methionine (M), leucine (L) or isoleucine (I) and maintain a consensus amino acid at the anchor residue positions of the peptide.

In general, it is preferred that fewer than all of the amino acids are changed when preparing variant polypeptides. Where particular amino acid residues are known to confer function, such amino acids will not be replaced, or alternatively, will be replaced by conservative amino acid substitutions. Preferably, 1, 2, 3, 4, 5, 6, 7, 8, and so on up to one fewer than the length of the peptide are changed when preparing variant polypeptides. It is generally preferred that the fewest number of substitutions is made. Thus, one method for generating variant polypeptides is to substitute all other amino acids for a particular single amino acid, then assay activity of the variant, then repeat the process with one or more of the polypeptides having the best activity.

As another example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by CTLs when bound to MHC. These variants can be tested for improved stability and are useful, inter alia, in vaccine compositions.

Conservative amino-acid substitutions in the amino acid sequence of CT antigen polypeptides to produce functionally equivalent variants of CT antigen polypeptides typically are made by alteration of a nucleic acid encoding a CT antigen polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a CT antigen polypeptide. Where amino acid substitutions are made to a small unique fragment of a CT antigen polypeptide, such as an antigenic epitope recognized by autologous or allogeneic sera or cytolytic T lymphocytes, the substitutions can be made by directly synthesizing the peptide. The activity of functionally equivalent fragments of CT antigen polypeptides can be tested by cloning the gene encoding the altered CT antigen polypeptide into a bacterial or mammalian expression vector, introducing the vector into an appropriate host cell, expressing the altered CT antigen polypeptide, and testing for a functional capability of the CT antigen polypeptides as disclosed herein. Peptides which are chemically synthesized can be tested directly for function, e.g., for binding to antisera recognizing associated antigens.

The invention also provides, in certain embodiments, “dominant negative” polypeptides derived from CT antigen polypeptides. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, given the teachings contained herein of CT antigens, especially those which are similar to known proteins which have known activities, one of ordinary skill in the art can modify the sequence of the CT antigens by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for diminution in a selected and/or for retention of such an activity. Other similar methods for creating and testing dominant negative variants of a protein will be apparent to one of ordinary skill in the art.

The invention as described herein has a number of uses, some of which are described elsewhere herein. First, the invention permits isolation of the CT antigen protein molecules. A variety of methodologies well-known to the skilled practitioner can be utilized to obtain isolated CT antigen molecules. The polypeptide may be purified from cells which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide. Translation of mRNA in cell-free extracts such as the reticulocyte lysate system also may be used to produce polypeptide. Those skilled in the art also can readily follow known methods for isolating CT antigen polypeptides. These include, but are not limited to, immunochromatography, HPLC, size-exclusion chromatography, ion-exchange chromatography and immune-affinity chromatography.

The invention also makes it possible isolate proteins which bind to CT antigens as disclosed herein, including antibodies and cellular binding partners of the CT antigens. Additional uses are described further herein.

The isolation and identification of CT antigen genes also makes it possible for the artisan to diagnose a disorder characterized by expression of CT antigens. These methods involve determining expression of one or more CT antigen nucleic acids, and/or encoded CT antigen polypeptides and/or peptides derived therefrom. In the former situation, such determinations can be carried out via any standard nucleic acid determination assay, including the polymerase chain reaction, or assaying with labeled hybridization probes. In the latter two situations, such determinations can be carried out by immunoassays including, for example, ELISAs for the CT antigens, immunohistochemistry on tissue samples, and screening patient antisera for recognition of the polypeptide.

The invention further includes nucleic acid or protein microarrays with CT antigens or nucleic acids encoding such polypeptides. In this aspect of the invention, standard techniques of microarray technology are utilized to assess expression of the CT antigens and/or identify biological constituents that bind such polypeptides. The constituents of biological samples include antibodies, lymphocytes (particularly T lymphocytes), and the like. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. Nucleic acid arrays, particularly arrays that bind CT antigens, also can be used for diagnostic applications, such as for identifying subjects that have a condition characterized by CT antigen expression.

Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate.

Targets are peptides or proteins and may be natural or synthetic. The tissue may be obtained from a subject or may be grown in culture (e.g. from a cell line).

In some embodiments of the invention one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

In other embodiments, one or more control peptide or nucleic acid molecules are attached to the substrate. Preferably, control nucleic acid molecules allow determination of factors such as binding characteristics, reagent quality and effectiveness, hybridization success, and analysis thresholds and success.

Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol.21, January 1999, the entire contents of which is incorporated by reference herein.

According to the present invention, nucleic acid microarray substrates may include but are not limited to glass, silica, aluminosilicates, borosilicates, metal oxides such as alumina and nickel oxide, various clays, nitrocellulose, or nylon. In all embodiments a glass substrate is preferred. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. In one embodiment, preferred probes are sets of two or more of the CT antigen nucleic acid molecules set forth herein. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or olignucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium. In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets for microarrays are nucleic acids selected from the group, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g. from a cell line).

In embodiments of the invention one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

In some embodiments, one or more control peptide or nucleic acid molecules are attached to the substrate. Preferably, control nucleic acid molecules allow determination of factors such as binding characteristics, reagent quality and effectiveness, hybridization success, and analysis thresholds and success.

Expression of CT antigen polypeptides can also be determined using protein measurement methods. Preferred methods of specifically and quantitatively measuring proteins include, but are not limited to: mass spectroscopy-based methods such as surface enhanced laser desorption ionization (SELDI; e.g., Ciphergen ProteinChip System, Ciphergen Biosystems, Fremont CA), non-mass spectroscopy-based methods, and immunohistochemistry-based methods such as two-dimensional gel electrophoresis.

SELDI methodology may, through procedures known to those of ordinary skill in the art, be used to vaporize microscopic amounts of tumor protein and to create a “fingerprint” of individual proteins, thereby allowing simultaneous measurement of the abundance of many proteins in a single sample. Preferably SELDI-based assays may be utilized to classify tumor samples with respect to the expression of a variety of CT antigens. Such assays preferably include, but are not limited to the following examples. Gene products discovered by RNA microarrays may be selectively measured by specific (antibody mediated) capture to the SELDI protein disc (e.g., selective SELDI). Gene products discovered by protein screening (e.g., with 2-D gels), may be resolved by “total protein SELDI” optimized to visualize those particular markers of interest from among CT antigens.

Tumors can be classified based on the measurement of multiple CT antigens. Classification based on CT antigen expression can be used to stage disease, monitor progression or regression of disease, and select treatment strategies for the cancer patients.

The invention also involves agents such as polypeptides which bind to CT antigen polypeptides. Such binding agents can be used, for example, in screening assays to detect the presence or absence of CT antigen polypeptides and complexes of CT antigen polypeptides and their binding partners and in purification protocols to isolated CT antigen polypeptides and complexes of CT antigen polypeptides and their binding partners. Such agents also can be used to inhibit the native activity of the CT antigen polypeptides, for example, by binding to such polypeptides.

The invention, therefore, embraces peptide binding agents which, for example, can be antibodies or fragments of antibodies having the ability to selectively bind to CT antigen polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. See, e.g., U.S. Pat. Nos. 5,545,806, 6,150,584, and references cited therein. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Accordingly, the invention involves polypeptides of numerous size and type that bind specifically to CT antigen polypeptides, and complexes of both CT antigen polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the CT antigen polypeptide. This process can be repeated through several cycles of reselection of phage that bind to the CT antigen polypeptide. Repeated rounds lead to enrichment of phage bearing particular sequences. DNA sequence analysis can be conducted to identify the sequences of the expressed polypeptides. The minimal linear portion of the sequence that binds to the CT antigen polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the CT antigen polypeptides. Thus, the CT antigen polypeptides of the invention, or a fragment thereof, can be used to screen peptide libraries, including phage display libraries, to identify and select peptide binding partners of the CT antigen polypeptides of the invention. Such molecules can be used, as described, for screening assays, for purification protocols, for interfering directly with the functioning of CT antigen and for other purposes that will be apparent to those of ordinary skill in the art.

As detailed herein, the foregoing antibodies and other binding molecules may be used for example to identify tissues expressing protein or to purify protein. Antibodies also may be coupled to specific diagnostic labeling agents for imaging of cells and tissues that express CT antigens or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium. Other diagnostic agents useful in the invention will be apparent to one of ordinary skill in the art.

As used herein, “therapeutically useful agents” include any therapeutic molecule which desirably is targeted selectively to a cell expressing one of the cancer antigens disclosed herein, including antineoplastic agents, radioiodinated compounds, toxins, other cytostatic or cytolytic drugs, and so forth. Antineoplastic therapeutics are well known and include: aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon-α, lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60.

In some embodiments, antibodies prepared according to the invention are specific for complexes of MHC molecules and the CT antigens described herein.

When “disorder” is used herein, it refers to any pathological condition where the CT antigens are expressed. An example of such a disorder is cancer, including but not limited to: biliary tract cancer; bladder cancer; breast cancer; brain cancer including glioblastomas and medulloblastomas; cervical cancer; choriocarcinoma; colon cancer; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

Samples of tissue and/or cells for use in the various methods described herein can be obtained through standard methods such as tissue biopsy, including punch biopsy and cell scraping, and collection of blood or other bodily fluids by aspiration or other methods.

In certain embodiments of the invention, an immunoreactive cell sample is removed from a subject. By “immunoreactive cell” is meant a cell which can mature into an immune cell (such as a B cell, a helper T cell, or a cytolytic T cell) upon appropriate stimulation. Thus immunoreactive cells include CD34⁺ hematopoietic stem cells, immature T cells and immature B cells. When it is desired to produce cytolytic T cells which recognize a CT antigen, the immunoreactive cell is contacted with a cell which expresses a CT antigen under conditions favoring production, differentiation and/or selection of cytolytic T cells; the differentiation of the T cell precursor into a cytolytic T cell upon exposure to antigen is similar to clonal selection of the immune system.

Some therapeutic approaches based upon the disclosure are premised on a response by a subject's immune system, leading to lysis of antigen presenting cells, such as cancer cells which present one or more CT antigens. One such approach is the administration of autologous CTLs specific to a CT antigen/MHC complex to a subject with abnormal cells of the phenotype at issue. It is within the ability of one of ordinary skill in the art to develop such CTLs in vitro. An example of a method for T cell differentiation is presented in International Application number PCT/US96/05607. Generally, a sample of cells taken from a subject, such as blood cells, are contacted with a cell presenting the complex and capable of provoking CTLs to proliferate. The target cell can be a transfectant, such as a COS cell. These transfectants present the desired complex of their surface and, when combined with a CTL of interest, stimulate its proliferation. COS cells are widely available, as are other suitable host cells. Specific production of CTL clones is well known in the art. The clonally expanded autologous CTLs then are administered to the subject.

Another method for selecting antigen-specific CTL clones has recently been described (Altman et al., Science 274:94-96, 1996; Dunbar et al., Curr. Biol. 8:413-416, 1998), in which fluorogenic tetramers of MHC class I molecule/peptide complexes are used to detect specific CTL clones. Briefly, soluble MHC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g. phycoerythrin) at a molar ratio or 4:1. Tetramers are then contacted with a source of CTLs such as peripheral blood or lymph node. The tetramers bind CTLs which recognize the peptide antigen/MHC class I complex. Cells bound by the tetramers can be sorted by fluorescence activated cell sorting to isolate the reactive CTLs. The isolated CTLs then can be expanded in vitro for use as described herein.

To detail a therapeutic methodology, referred to as adoptive transfer (Greenberg, J. Immunol. 136(5): 1917, 1986; Riddel et al., Science 257: 238, 1992; Lynch et al, Eur. J. Immunol. 21: 1403-1410,1991; Kast et al., Cell 59: 603-614, 1989), cells presenting the desired complex (e.g., dendritic cells) are combined with CTLs leading to proliferation of the CTLs specific thereto. The proliferated CTLs are then administered to a subject with a cellular abnormality which is characterized by certain of the abnormal cells presenting the particular complex. The CTLs then lyse the abnormal cells, thereby achieving the desired therapeutic goal.

The foregoing therapy assumes that at least some of the subject's abnormal cells present the relevant HLA/CT antigen complex. This can be determined very easily, as the art is very familiar with methods for identifying cells which present a particular HLA molecule, as well as how to identify cells expressing DNA of the pertinent sequences, in this case a CT antigen sequence. Once cells presenting the relevant complex are identified via the foregoing screening methodology, they can be combined with a sample from a patient, where the sample contains CTLs. If the complex presenting cells are lysed by the mixed CTL sample, then it can be assumed that a CT antigen is being presented, and the subject is an appropriate candidate for the therapeutic approaches set forth supra.

Adoptive transfer is not the only form of therapy that is available in accordance with the invention. CTLs can also be provoked in vivo, using a number of approaches. One approach is the use of non-proliferative cells expressing the complex. The cells used in this approach may be those that normally express the complex, such as irradiated tumor cells or cells transfected with one or both of the genes necessary for presentation of the complex (i.e. the antigenic peptide and the presenting HLA molecule). Chen et al. (Proc. Natl. Acad. Sci. USA 88: 110-114,1991) exemplifies this approach, showing the use of transfected cells expressing HPVE7 peptides in a therapeutic regime. Various cell types may be used. Similarly, vectors carrying one or both of the genes of interest may be used. Viral or bacterial vectors are especially preferred. For example, nucleic acids which encode a CT antigen polypeptide or peptide may be operably linked to promoter and enhancer sequences which direct expression of the CT antigen polypeptide or peptide in certain tissues or cell types. The nucleic acid may be incorporated into an expression vector. Expression vectors may be unmodified extrachromosomal nucleic acids, plasmids or viral genomes constructed or modified to enable insertion of exogenous nucleic acids, such as those encoding CT antigen, as described elsewhere herein. Nucleic acids encoding a CT antigen also may be inserted into a retroviral genome, thereby facilitating integration of the nucleic acid into the genome of the target tissue or cell type. In these systems, the gene of interest is carried by a microorganism, e.g., a Vaccinia virus, pox virus, herpes simplex virus, retrovirus or adenovirus, and the materials de facto “infect” host cells. The cells which result present the complex of interest, and are recognized by autologous CTLs, which then proliferate.

A similar effect can be achieved by combining the CT antigen or an immune response stimulatory fragment thereof with an adjuvant to facilitate incorporation into antigen presenting cells in vivo. The CT antigen polypeptide is processed to yield the peptide partner of the HLA molecule while a CT antigen peptide may be presented without the need for further processing. Generally, subjects can receive an intradermal injection of an effective amount of the CT antigen. Initial doses can be followed by booster doses, following immunization protocols standard in the art.

The invention involves the use of various materials disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. Generally accepted animal models can be used for testing of immunization against cancer using a CT antigen nucleic acid. For example, human cancer cells can be introduced into a mouse to create a tumor, and one or more CT antigen nucleic acids can be delivered by the methods described herein. The effect on the cancer cells (e.g., reduction of tumor size) can be assessed as a measure of the effectiveness of the CT antigen nucleic acid immunization. Of course, testing of the foregoing animal model using more conventional methods for immunization include the administration of one or more CT antigen polypeptides or peptides derived therefrom, optionally combined with one or more adjuvants and/or cytokines to boost the immune response. Methods for immunization, including formulation of a vaccine composition and selection of doses, route of administration and the schedule of administration (e.g. primary and one or more booster doses), are well known in the art. The tests also can be performed in humans, where the end point is to test for the presence of enhanced levels of circulating CTLs against cells bearing the antigen, to test for levels of circulating antibodies against the antigen, to test for the presence of cells expressing the antigen and so forth.

As part of the immunization compositions, one or more CT antigens or stimulatory fragments thereof are administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham); QS-7, QS-17, QS-18, and QS-L1 (So et al., Mol. Cells 7:178-186, 1997); incomplete Freund's adjuvant; complete Freund's adjuvant; montanide; immunostimulatory oligonucleotides (see e.g. CpG oligonucleotides described by Kreig et al., Nature 374:546-9, 1995); vitamin E and various water-in-oil emulsions prepared from biodegradable oils such as squalene and/or tocopherol. Preferably, the peptides are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of peptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268:1432-1434, 1995), GM-CSF and IL-18. Thus cytokines can be administered in conjunction with antigens and adjuvants to increase the immune response to the antigens.

There are a number of immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng P., et al. Proc. Natl. Acad. Sci. USA 95 (11):6284-6289 (1998)).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., J. Immunol, 154:5637-5648 (1995)). Tumor cell transfection with B7 has been discussed in relation to in vitro CTL expansion for adoptive transfer immunotherapy by Wang et al., (J. Immunol., 19:1-8 (1986)). Other delivery mechanisms for the B7 molecule would include nucleic acid (naked DNA) immunization (Kim J., et al. Nat Biotechnol., 15:7:641-646 (1997)) and recombinant viruses such as adeno and pox (Wendtner et al., Gene Ther., 4:7:726-735 (1997)). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642 (1997), Fenton et al., J. Immunother., 21:2:95-108 (1998)).

Lymphocyte function associated antigen-i (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., J. Immunother., 21:2:95-108 (1998)). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature, 393:474 (1998), Bennett et al., Nature, 393:478 (1998), Schoenberger et al., Nature, 393:480 (1998)). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor antigens which are normally encountered outside of a inflammatory context or are presented by non-professional APCs (tumor cells). In these situations Th help and B7 costimulation signals are not provided. This mechanism might be used in the context of antigen pulsed DC based therapies or in situations where Th epitopes have not been defined within known TRA precursors.

A CT antigen polypeptide, or a fragment thereof, also can be used to isolate their native binding partners. Isolation of such binding partners may be performed according to well-known methods. For example, isolated CT antigen polypeptides can be attached to a substrate (e.g., chromatographic media, such as polystyrene beads, or a filter), and then a solution suspected of containing the binding partner may be applied to the substrate. If a binding partner which can interact with CT antigen polypeptides is present in the solution, then it will bind to the substrate-bound CT antigen polypeptide. The binding partner then may be isolated.

It will also be recognized that the invention embraces the use of the CT antigen cDNA sequences in expression vectors, as well as to transfect host cells and cell lines, be these prokaryotic (e.g., E. coli), or eukaryotic (e.g., dendritic cells, B cells, CHO cells, COS cells, yeast expression systems and recombinant baculovirus expression in insect cells). Especially useful are mammalian cells such as human, mouse, hamster, pig, goat, primate, etc. They may be of a wide variety of tissue types, and include primary cells and cell lines. Specific examples include keratinocytes, peripheral blood leukocytes, bone marrow stem cells and embryonic stem cells. The expression vectors require that the pertinent sequence, i.e., those nucleic acids described supra, be operably linked to a promoter.

The invention also contemplates delivery of nucleic acids, polypeptides or peptides for vaccination. Delivery of polypeptides and peptides can be accomplished according to standard vaccination protocols which are well known in the art. In another embodiment, the delivery of nucleic acid is accomplished by ex vivo methods, i.e. by removing a cell from a subject, genetically engineering the cell to include a CT antigen, and reintroducing the engineered cell into the subject. One example of such a procedure is the use of dendritic cells as delivery and antigen presentation vehicles for the administration of CT antigens in vaccine therapies. Another example of such a procdure is outlined in U.S. Pat. No. 5,399,346 and in exhibits submitted in the file history of that patent, all of which are publicly available documents. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

In preferred embodiments, a virus vector for delivering a nucleic acid encoding a CT antigen is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In preferred embodiments, the virus vector is an adenovirus or an alphavirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

Preferably the foregoing nucleic acid delivery vectors: (1) contain exogenous genetic material that can be transcribed and translated in a mammalian cell and that can induce an immune response in a host, and (2) contain on a surface a ligand that selectively binds to a receptor on the surface of a target cell, such as a mammalian cell, and thereby gains entry to the target cell.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection or infection with the foregoing viruses including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies which selectively bind a CT antigen, alone or as a complex with a MHC molecule. Especially preferred are monoclonal antibodies. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

When administered, the therapeutic compositions of the present invention can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resort to undue experimentation. When using antisense preparations of the invention, slow intravenous administration is preferred.

The compositions of the invention are administered in effective amounts. An “effective amount” is that amount of a CT antigen composition that alone, or together with further doses, produces the desired response, e.g. increases an immune response to the CT antigen. In the case of treating a particular disease or condition characterized by expression of one or more CT antigens, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of CT antigen or nucleic acid encoding CT antigen for producing the desired response in a unit of weight or volume suitable for administration to a patient. The response can, for example, be measured by determining the immune response following administration of the CT antigen composition via a reporter system by measuring downstream effects such as gene expression, or by measuring the physiological effects of the CT antigen composition, such as regression of a tumor or decrease of disease symptoms. Other assays will be known to one of ordinary skill in the art and can be employed for measuring the level of the response.

The doses of CT antigen compositions (e.g., polypeptide, peptide, antibody, cell or nucleic acid) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, for treatments for eliciting or increasing an immune response, doses of CT antigen are formulated and administered in doses between 1 ng and 1 mg, and preferably between 10 ng and 100 μg, according to any standard procedure in the art. Where nucleic acids encoding CT antigen or variants thereof are employed, doses of between 1 ng and 0.1 mg generally will be formulated and administered according to standard procedures. Other protocols for the administration of CT antigen compositions will be known to one of ordinary skill in the art, in which the dose amount, schedule of injections, sites of injections, mode of administration (e.g., intra-tumoral) and the like vary from the foregoing. Administration of CT antigen compositions to mammals other than humans, e.g. for testing purposes or veterinary therapeutic purposes, is carried out under substantially the same conditions as described above.

Where CT antigen peptides are used for vaccination, modes of administration which effectively deliver the CT antigen and adjuvant, such that an immune response to the antigen is increased, can be used. For administration of a CT antigen peptide in adjuvant, preferred methods include intradermal, intravenous, intramuscular and subcutaneous administration. Although these are preferred embodiments, the invention is not limited by the particular modes of administration disclosed herein. Standard references in the art (e.g., Remington 's Pharmaceutical Sciences, 18th edition, 1990) provide modes of administration and formulations for delivery of immunogens with adjuvant or in a non-adjuvant carrier.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A CT antigen composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of CT antigen polypeptides or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington 's Pharmaceutical Sciences, Mack Publishing Co., Easton, Pa.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. An isolated nucleic acid as used herein is not a naturally occurring chromosome.

As used herein with respect to polypeptides, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

### EXAMPLES

### Example 1

### Identification of CT Antigens

Much attention has been given to the potential of CT antigens as targets for cancer vaccine development, and, other than mutational antigens and virus encoded antigens, they clearly represent the most specific tumor antigens discovered to date. However, the CT antigens also provide a new way to think about cancer and its evolution during the course of the disease.

The starting point for this view is the fact that CT antigen expression is restricted to early germ cell development and cancer. Germ cells give rise to gametes (oocytes and spermatocytes) and trophoblastic cells that contribute to the formation of the chorion and the placenta. Primitive germ cells arise in the wall of the yolk sack and during embryogenesis migrate to the future site of the gonads. In oogenesis, the process begins before birth, with oogonia differentiating into primary oocytes. The primary oocytes, which reach their maximal numbers during fetal development, are arrested at the initial phase of meiosis, and do not renew and complete meiosis until ovulation and fertilization. In contrast, spermatogenesis begins at puberty and is a continuous process of mitosis to maintain the spermatogonia pool and meiosis to generate the mature sperm population. CT antigens, like SCP-1 and OY-TES-1, the proacrosomal binding protein precursor, are clearly important in gametogenesis, and it is likely that the other CT antigens with their restricted expression in gametes and trophoblasts also play a critical role in early germ cell development.

One possibility to account for aberrant CT expression in cancer relates to the global demethylation associated with certain cancers (42). The promoter region of the MAGE gene has binding sites for transcriptional activators and these sites are methylated in normal somatic cells but demethylated in MAGE-expressing cancer cells and testis. Although cancer-associated demethylation could therefore account for CT (MAGE) expression in tumors, it does not easily accommodate the usual observation of non-coordinate expression patterns (sets) of different CT antigens in most tumors. Also, the marked heterogeneity in CT expression in some tumors (34, 43) is also not easily explicable by a global demethylation process.

Another mechanism for reactivating CT expression in cancer has to do with mutations in regulatory regions of the CT genes. Although no mutations in CT genes have been found to date, more extensive sequencing, particularly in the promoter region, needs to be done before this can be excluded. However, mutation of CT genes is unlikely to be a common mechanism for the induction of CT expression in cancer.

Another possibility to account for the appearance of CT antigens in cancer is the induction or activation of a gametogenic program in cancer. According to this view, the different CT sets seen in cancer would replicate the corresponding sets of CT antigens normally expressed during different stages of gametogenesis or trophoblast development. Triggering events for inducing the gametogenic program could be a mutation in an as yet unidentified master switch in germ cell development, or an activation of this master switch by threshold mutations in oncogenes, suppressor genes, or other genes in cancer. It is also possible that activation of a single CT gene could be the switch for activating other genes in the gametogenic program. Supporting evidence for this idea comes from the study of synovial sarcoma, where a translocation event involving the SYT gene on chromosome 18 and the SSX-1 or SSX-2 gene on chromosome X is associated with high expression of unrelated CT antigens, such as NY-ESO-1 and MAGE (44, 45). Extending this line of reasoning and relating it to the role of demethylation in the appearance of CT antigens, a demethylation state in cancer (whatever its cause) could induce the gametogenic program and result in the activation of silent CT genes. Alternatively, demethylation may be an intrinsic part of the gametogenic program and therefore a consequence, not a cause, of switching on the gametogenic program and CT genes in cancer.

In addition to questions about mechanisms for reactivating CT antigen expression in cancer, another important issue is whether expression of these genes in the cancer cell contributes to its malignant behavior. The finding that gametes, trophoblasts and cancers share a battery of antigens restricted to these cell types suggests extending the search for other shared characteristics.

It was a similarity in the biological features of trophoblasts and cancer cells that prompted the Scottish embryologist John Beard at the turn of the last century to propose his trophoblastic theory of cancer (46, 47). In his view, cancers arise from germ cells that stray or are arrested in their trek to the gonads. Under the influence of carcinogenic stimuli, such cells undergo a conversion to malignant trophoblastic cells. These malignant trophoblastic cells take on features of the resident cell types in different organs, but the resulting cancers, no matter their site of origin or how distinct they appear morphologically, are of trophoblastic origin. Beard ascribed the invasive, destructive and metastatic features of cancer to functions normally displayed by trophoblastic cells, e.g., invasion of blood vessels, growth into the uterine wall, and spread beyond the uterus. From a contemporary perspective, Beard's idea that cancers are derived from arrested germ cells seems incompatible with our growing knowledge of serological and molecular markers that distinguish different pathways of normal differentiation and their preservation in cancer. Beard's insight that trophoblasts and cancer cells share common features is better explained by the induction of a gametogenic program in resident cancer cells, rather than the derivation of cancer from an aberrant germ cell. The end result, however, would be the same—selected features of cells undergoing gametogenesis and trophoblast development being imposed on transformed somatic cells.

In addition to CT antigens, other features shared by germ cells and cancer are identified. For example, SCP-1, a critical element in the meiotic program, is expressed in non-germ cell cancers. The induction of a meiotic program in a somatic cell, normal or malignant, likely leads to chromosomal anarchy, a prime feature of advanced cancers. Accordingly, other proteins uniquely associated with meiosis and expressed in cancer cells also are identified as candidate CT antigens.

OY-TES-1, the proacrosin binding protein precursor that is part of the unique program leading to the formation of spermatozoa, has been identified as a CT antigen. Accordingly, other mature sperm-specific gene products that are expressed in cancer cells also are identified as candidate CT antigens.

In addition, expression of CT antigens by trophoblasts sheds new light on an old issue—the much studied sporadic production of human chorionic gonadotropin (HCG) and other trophoblastic hormones by human cancers (e.g., 48, 49, 50). The production of HCG by cancer cells has been generally viewed as yet another indication of the genetic instability of cancer cells, resulting in the random and aberrant activation of silent genes during carcinogenesis and tumor progression. However, it can also be viewed as a consequence of the induction of a gametogenic/trophoblastic program in cancer, one that would also result in the semi-coordinate expression of CT antigens. Activation of this program would also confer other properties of germ cells, gametes, and trophoblasts on cancer cells, but these are more difficult to relate in any precise fashion. Nonetheless, immortalization, invasion, lack of adhesion, migratory behavior, induction of blood vessels, demethylation, and downregulation of MHC, are some features shared by cancer and by cells undergoing germ cell/gamete/trophoblast differentiation pathways. The metastatic properties of cancer may also have counterparts in the migratory behavior of germ cells, and in the propensity of normal trophoblast cells to migrate to other organs, such as the lung, during normal pregnancy, but then to undergo involution at term.

In pursing the idea of a program change in cancer leading to the expression of gametogenic features, a hypothesis termed “Gametogenic Program Induction in Cancer” (GPIC), it might be well to distinguish at least four different pathways involved in germ cell development: A) germ cell→germ cell, B) germ cell→oogonia→oocytes, C) germ cell→spermatogonia→sperm, and D) germ cell→trophoblast. The meiotic program would be common to B and C, proteins like OY-TES-1 would be restricted to C, and HCG would be a characteristic of D. The reason for distinguishing these pathways and ultimately stages in each pathway is that the variety of patterns or sets of CT antigens observed in different cancers may be a reflection of the germ cell program, e.g., pathway and stage that has been induced in these cancers.

With this background and framework of thinking about the relation of gametogenesis and cancer development, there are a number of approaches to be taken to identify additional CT antigens.

1. The search for new CT antigens is accomplished using several methodologies, including SEREX (see, for example, ref. 10), particularly with libraries from testis, normal or malignant trophoblasts, or tumors or tumor cell lines (growing with or without demethylating agents) that express a range of CT antigens, and by extending the use of representational difference analysis. Bioinformatics and chip technology are used for mining databanks for transcripts that show cancer/gamete/trophoblast specificity (e.g., screening annotation of sequence records).

2. The expression pattern of known CT antigens in normal gametogenesis and trophoblast development is determined to identify markers that distinguish different pathways and stages in the normal gametogenic program. This information provides a basis for interpreting the complex patterns of CT expression in cancers in relation to gametogenic pathways/stages, and provides new ways to classify cancer on the basis of CT phenotypes.

3. The frequency of expression of individual CT antigens in different tumor types has been defined for those CT antigens known to date. In addition to analyzing frequency of expression for CT antigens identified by the methods described herein, additional information is gathered about the composite CT phenotype of individual tumors, and how frequently these composite CT patterns are seen in tumors of different origin. Databases of clinical, genotypic, phenotypic and CT antigen expression data for individual tumors are established to compare the properties of individual tumors and establish correlations between the data. With this information, correlations of CT expression with other biological features of the tumor, e.g., growth rate, local vs. invasive, primary vs. metastatic, different metastatic deposits in the same patient, etc. can be established.

4. Determining which stage in the life history of cancer that CT (gametogenic) features are induced can be approached in model systems in the mouse, in vitro systems with human cells, or with naturally occurring tumors in man that show incremental stages in tumor progression. As discussed above, there is evidence that CT expression is a sign of greater malignancy.

5. The heterogeneous expression of CT antigens in a large proportion of human cancers needs to be understood. This may reflect a quantitative difference in levels of mRNA/protein in CT⁺ and CT⁻ cells, or there may be a qualitative distinction between CT⁺ and CT⁻ cells in CT mRNA/protein expression. Laser dissection microscopy may be one way to analyze this question and cloning of tumor cells from a tumor with heterogeneous CT expression is another approach to understand heterogeneous expression. There is a growing impression that established human cancer cell lines show a higher frequency of CT antigen expression than what would be expected from CT typing of the corresponding tumor type, particularly tumors with a low frequency of CT expression. This could be a secondary consequence of in vitro culture, or it could be that CT+cells (even if they represent only a minority population of the tumor) have a growth advantage for propagating in vitro, and possibly also in vivo.

6. Although CT antigens provide a strong link between the gametogenic program and cancer, it is determined whether other distinguishing features of gamete development are expressed by cancer and whether their expression is correlated with CT antigen expression. The many reports over the last three decades of HCG production by certain human cancers provides a specific starting point to explore this issue and ask whether the production of HCG is correlated with CT antigen expression, particularly a unique pattern of CT expression, such as a pattern reflecting the trophoblast program.

7. Transgenic and knock-out approaches using mouse CT counterparts, and transfection analysis with CT coding genes in normal and malignant human cells are performed to define the role of CT antigens in gametogenesis and trophoblast development and their functional significance in cancer.

### Example 2

### Identification of CT Gene Products

In order to identify new CT gene products, the Unigene database, a compilation of both EST and Genbank databases, was mined for transcripts expressed exclusively in cancer and normal testis. Subsequent RT-PCR analysis of candidate transcripts identified several gene products with highly restricted mRNA expression patterns, including three newly defined CT genes.

Methods and Materials

Bioinformatic Identification of Cancer/Testis-Associated Unigene Clusters

The cDNA X profiler tool of the Cancer Genome Anatomy Project (http://cgap.nci.nih.gov/Tissues/xProfiler) was used to search the Unigene database in the following manner. First, 2 pools of expressed sequence tags (ESTs) were established. Pool A consisted of ESTs derived from 6 normal testis cDNA libraries, and Pool B consisted of ESTs derived from 188 tumor-derived cDNA libraries (all histological types). The X profiler search engine was directed to identify those Unigene clusters containing ESTs from both Pool A and Pool B, and exclude Unigene clusters containing ESTs from any other normal tissue cDNA library. Tissue expression patterns of the resultant Unigene clusters were also analyzed by in silico Serial Analysis of Gene Expression (SAGE) using the SAGE:Gene to tag mapping tool associated with each Unigene cluster entry. Their relation to known gene products was determined by BLAST searches of nucleotide and protein databases (http://www.ncbi.nlm.nih.gov/BLAST/) or by motif analysis of putative protein translations (http://motif.genome.ad jp). Furthermore, BLAST searches of representative ESTs from all identified Unigene clusters were performed against the human genome sequence database in order to obtain gene mapping information and to determine intron/exon boundaries used in PCR primer design.

Reverse Transcriptase—PCR (RT-PCR) Analysis

Total RNA from 20 different normal human tissues was purchased from Clontech Laboratories Incorporated (Palo Alto, Calif.) and Ambion Incorporated (Austin, Tex.). Tumor tissues were derived from surgical specimens obtained from Memorial Sloan-Kettering Cancer Center, Weill Medical College of Cornell University and Krankenhaus Nordwest (Frankfurt, Germany). Total RNA from tumor tissues was prepared by the guanidinium thiocyanate method.

The cDNA preparations used as templates in the RT-PCR reactions were prepared using the Superscript first strand synthesis kit (Invitrogen Life Technologies, Carlsbad, Calif.). The cDNA was synthesized by incubating 5 μg of total RNA in 40 μl of 1× reverse transcriptase buffer containing 100 ng random hexamers, 0.5 mM dNTP, 5.0 mM MgCl₂, 10 μM DTT, 80 U ribonuclease inhibitor and 100 U Superscript II reverse transcriptase at 42° C. for 50 min. Control templates for assessing amplification of genomic DNA were prepared as duplicate samples lacking reverse transcriptase.

Oligonucleotide primers, homologous to ESTs present in selected Unigene clusters, were synthesized commercially (Invitrogen Life Technologies). DNA sequences of relevant primer pairs are provided below.

| | |
|---|---|
| CT15 forward: | |
| 5′AGGAATTATGAAACCACTTG | (SEQ ID NO:1) |
| CT15 reverse: | |
| 5′GACAACAGTTGTATCAGACC | (SEQ ID NO:2) |
| CT16 forward: | |
| 5′CAAGGAGAGGTCGTGTCTTCG | (SEQ ID NO:3) |
| CT16 reverse: | |
| 5′GGATCTTGTTACATGCTCACTCATATC | (SEQ ID NO:4) |
| CT16.2 forward: | |
| 5′CCAGATTAAGAATAACGTTC | (SEQ ID NO:5) |
| CT16.2 reverse: | |
| 5′AGAGGAAATGACCAAGAGTC | (SEQ ID NO:6) |
| CT17 forward: | |
| 5′ACAAGACTAGCTTATGTGTGG | (SEQ ID NO:7) |
| CT17 reverse: | |
| 5′TTGAGCAAGAATCTTGACTTC | (SEQ ID NO:8) |

RT-PCR was performed as follows. Twenty-five μl PCR reaction mixtures, consisting of 2 μl cDNA (or 2.0 μl of genomic DNA amplification controls), 0.2 mM dNTP, 1.5 mM MgCl₂, 0.25 μM gene specific forward and reverse primers, and 2.5 U Platinum Taq DNA polymerase (Invitrogen Life Technologies), were heated to 94° C. for 2 min., followed by 34 thermal cycles of 94° C. for 30 seconds, 55° C. for 30 seconds and 72° C. for 1 min., and a final cycle of 94° C. for 30 seconds, 55° C. for 30 and 72° C. for 5 min. Thermal cycling was performed using an ABI 7700 Sequence Detector. Resultant PCR products were analyzed in 2% Agarose/Tris-Acetate-EDTA gels and their identity was verified by DNA sequencing.

Real-Time Quantitative Reverse Transcription (RT)-PCR

Total RNA samples from 8 different normal adult tissues and 8 tumor specimens were prepared and reverse transcribed into cDNA as described above. Gene-specific TaqMan probes and PCR primers were designed using Primer Express software (PE Biosystems, Foster City Calif.) and synthesized commercially (PE Biosystems). DNA sequences of relevant taqman primer pairs and probes are provided below.

| | | |
|---|---|---|
| CT15taqman forward: | 5′GGGAGTATTGACAGTGGCAATTT | (SEQ ID NO:9) |
| CT15taqman reverse: | 5′TGTTCTCAATGTAGCGCCTTTC | (SEQ ID NO:10) |
| CT15taqman probe: | 5′CCACCTGTAGCTATACCAGCCAGACTCCC | (SEQ ID NO:11) |
| CT16taqman forward: | 5′GCAGAGTCCCCTCCCTGAC | (SEQ ID NO:12) |
| CT16taqman reverse: | 5′ACAGGAACTGGCTCTGCTTAAGA | (SEQ ID NO:13) |
| CT16taqman probe: | 5′TCAGGACCATCTCCAGGTGCATCCTC | (SEQ ID NO:14) |
| CT17taqman forward: | 5′CCAGAGTCTCATGTTAAAATCACTTACA | (SEQ ID NO:15) |
| CT17taqman reverse: | 5′GAAACACTTCCTCTCTTTCTTTAAGTACAA | (SEQ ID NO:16) |
| CT17taqman probe: | 5′ACCCAGAAAGACCACCACTTTGCAGGTA | (SEQ ID NO:17) |

Multiplex PCR reactions were prepared using 2.0 μl of cDNA (or 2.0 μl of genomic DNA amplification controls), diluted in TaqMan Universal PCR Master Mix supplemented with 200 nM Fam (6-carboxy-fluorescein) labeled gene-specific TaqMan probe, 300-900 nM gene specific forward and reverse primers (predetermined optimum concentration), and Vic labeled human beta glucuronidase, or phosphoglycero kinase endogenous control probe/primer mixtures (proprietary dye, PE Biosystems). Six 25 μl PCR reactions were prepared for each cDNA sample (3 per each endogenous control). PCR consisted of 40 cycles of 95° C. denaturation (15 seconds) and 60° C. annealing/extension (60 seconds). Thermal cycling and fluorescent monitoring were performed using an ABI 7700 sequence analyzer (PE Biosystems). The cycle interval at which a PCR product is first detected above a fixed threshold, termed cycle threshold (Ct), was determined for each sample, and the average of triplicate samples was recorded. The copy number of gene-specific transcripts per μg of RNA starting material was determined by comparison with a standard curve of Ct values generated from known concentrations of cDNA encoding the homologous gene product. To normalize the quantity of mRNA present in the total RNA samples, the Ct values obtained from the endogenous control were subtracted from the gene specific Ct values (ΔCt=Ct FAM−Ct VIC). Real-time RT-PCR of triplicate samples yielded two sets of three ΔCt values per each RNA sample (1 set per each endogenous control), and the mean of the six ΔCt values was calculated. The concentration of gene specific mRNA in normal or tumor-derived tissue, relative to normal testis was calculated by equating the normalized Ct values (ΔΔCt=ΔCt of normal or tumor tissue−ΔCt of normal testis), and determining the relative concentration (Relative Concentration=2^{-ΔΔCt}) The transcript copy number per μg of normalized total RNA was calculated by multiplying the mean relative concentration for each cDNA sample by the copy number in testicular tissue, which was determined from the standard curve (copy number=2^{-ΔΔCt}×copy number testis).

Results

Bioinformatic Analyses

1325 different Unigene clusters with in silico expression profiles resembling CT antigens were identified by mining the Unigene database for gene clusters containing expressed sequence tags (ESTs) derived exclusively from both tumor tissue and normal testis cDNA libraries. These cancer/testis-associated Unigene clusters represented 61 known genes and 1264 uncharacterized genes. As shown in Table 2, the Unigene clusters were placed into 2 categories. Group I consisted of 859 different gene clusters containing ESTs derived exclusively from testis and tumor cDNA libraries, termed cancer/testis (CT)-related Unigene clusters. Group II consisted of 400 different gene clusters containing ESTs derived exclusively from testis and germ cell tumors (but not other types of cancer), termed testis-related Unigene clusters. An additional 66 gene clusters were not pursued further since their respective Unigene database entries were modified during the course of this study, or literature reports of known gene products indicated they were expressed in other normal tissues, in addition to testis. In accordance with the Unigene database, the present study designates specific gene clusters as Homo sapiens.numerical description (e.g. Hs. 123456).

TABLE 2
In Silico classification of cancer/testis-associated Unigene clusters

| | | | | Number of |
|---|---|---|---|---|
| | | | | Unigene |
| Category of | | | | Clusters in |
| Unigene | | Sub- | Sub- Category | Each Sub- |
| Cluster | Category Description | Catagory | Description | Catagory |
| Group I | Cancer/Testis-related Unigene | IA | SAGE tags present only | 311 |
| | Clusters: contain ESTs only from | | in tumor and/or cell line | |
| | testis (or germ cell tumors) and | | SAGE libraries | |
| | tumor derived cDNA libraries | IB | No reliable SAGE tags | 265 |
| | | IC | SAGE tags present in | 283 |
| | | | normal tissue-derived | |
| | | | SAGE libraries | |
| Group II | Testis-related Unigene Clusters: | IIA | SAGE tags present only | 139 |
| | contain ESTs only from testis and | | in tumor and/or cell line | |
| | germ cell tumor cDNA libraries | | SAGE libraries | |
| | | IIB | No reliable SAGE tags | 171 |
| | | IIC | SAGE tags present in | 90 |
| | | | normal tissue-derived | |
| | | | SAGE libraries | |

The mRNA expression patterns of Group I and Group II Unigene clusters were further analyzed by in silico serial analysis of gene expression (SAGE). As shown in Table 2, group I and II Unigene clusters were further subdivided into subgroups A, B, and C, based on the presence and tissue distribution of homologous SAGE tags. Subgroups IA and IIA have SAGE tags that are present only in tumor and/or cell line derived SAGE libraries. Subgroups IB and IIB have no reliable SAGE tags. Subgroups IC and IIC have SAGE tags that are present in normal tissue SAGE libraries. Four known cancer testis antigens were identified among the 1325 Unigene clusters, including CT11p/Hs.293266 (Group IA), GAGE 4/Hs.183199 (Group IB), MAGEB1/Hs.73021 (Group IC), and SAGE/Hs.195292 (Group IIA).

Identification of Tissue-Restricted mRNA Transcripts by RT-PCR

The mRNA expression patterns of 73 of the 1325 Unigene clusters identified in the current study were analyzed by RT-PCR using a panel of RNA samples derived from 20 normal tissues. Several criteria were used for choosing these particular cancer/testis-associated Unigene clusters for RT-PCR analysis. Since a large proportion of known CT antigens map to chromosome X, all cancer/testis-associated Unigene clusters mapping to chromosome X were tested (19 total). Also, since melanoma and sarcoma express a large number of CT antigens, 10 cancer/testis-associated Unigene clusters having ESTs derived from melanoma or sarcoma libraries were tested. Those cancer/testis-associated Unigene clusters having functional significance in relation to cancer (e.g., transcription factors, adhesion molecules) were also tested (21 total). The remaining 23 Unigene clusters analyzed were chosen at random. In relation to cancer/testis-associated Unigene cluster sub-groupings, 59 CT-related Unigene clusters (38 from Group IA, 9 from Group IB, 12 from Group IC) and 14 testis-related Unigene clusters (6 from Group IIA, 7 from Group IIB and 1 from Group IIC) were analyzed by RT-PCR.

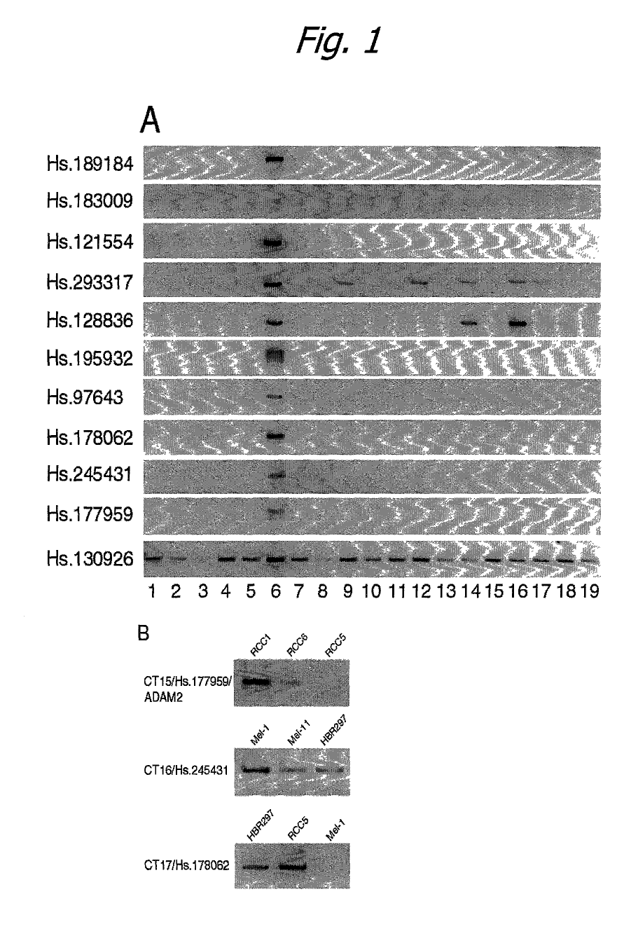

As shown in FIG. 1, ten of the 73 Unigene clusters analyzed by RT-PCR were considered differentially expressed, with transcripts detected in a limited number of normal tissues, i.e., mRNA expression detected in less than 7/20 normal tissues, and are listed in Table 3.

TABLE 3
Cancer/Testis (CT)-associated Unigene clusters identified by
database mining having restricted expression profiles in
normal tissues as determined by conventional RT-PCR

| | Category of CT- | | |
|---|---|---|---|
| Unigene | associated Unigene | Expression | Gene Product |
| cluster | Cluster (Table 2) | Profiles¹ | Description |
| Hs.121554 | IA | Testis only | An uncharacterized |
| | | | gene product with |
| | | | homology to |
| | | | members of the |
| | | | cystatin family of |
| | | | protease inhibitors |
| Hs.183009 | IA | Testis and brain | Regulatory factor X, |
| | | | 4 (RFX4) |
| Hs.178062 | IA | Testis only | An uncharacterized |
| | | | gene product with |
| | | | homology to |
| | | | phospholipase A1 |
| Hs.245431 | IB | Testis only | An uncharacterized |
| | | | gene product with |
| | | | homology to GAGE |
| | | | genes |
| Hs.177959 | IC | Testis only | A disintegrin and |
| | | | metalloproteinase 2 |
| | | | (ADAM2/fertilin β) |
| Hs.97643 | IC | Testis only | An uncharacterized |
| | | | gene product termed |
| | | | testis-specific |
| | | | protein TSP-NY |
| Hs.128836 | IC | Testis, ovary, | An uncharacterized |
| | | lung, cervix, | gene product with |
| | | | no protein motifs |
| | | | or similarities |
| Hs.195932 | IIA | Testis only² | An uncharacterized |
| | | | gene product termed |
| | | | testis transcript |
| | | | Y 12 (TTY12) |
| Hs.293317 | IIA | Testis, prostate, | An uncharacterized |
| | | ovary, lung, | gene product with |
| | | colon, breast | homology to GAGE |
| | | | genes |
| Hs.189184 | IIA | Testis only | Ubiquilin 3 |
| | | | (Ubqln 3) |

¹Normal tissue RNA panel included brain, testis, kidney, liver, pancreas, placenta, small intestine, heart, prostate, adrenal gland, spleen, fetal brain, colon, stomach, lung, bladder, ovary, breast, cervix and skeletal muscle
²Gene maps to chromosome Y. For this reason, RNA samples derived solely from female donors (kidney, colon, bladder, placenta, ovary, breast, cervix) are not applicable.

The mRNA expression patterns of the remaining 63 gene products were ubiquitously expressed in normal tissues (43 Unigene gene clusters), or yielded ambiguous RT-PCR results resulting from amplification of intronless DNA (8 Unigene gene clusters), non-specific amplification (4 Unigene gene clusters), or could not be amplified (8 Unigene gene clusters). Of the 10 differentially expressed transcripts identified, 7 were expressed only in testis (0/19 other normal tissue), and 3 other gene products were detected in a limited number of normal tissues besides testis and ovary (FIG. 1A). Of the 7 testis-restricted transcripts, 2 encode known proteins, Ubiquilin 3 (Ubqln 3, Hs. 189184, Group IIA) and disintegrin and metalloproteinase 2 or fertilin β (ADAM2, Hs.177959, Group IC) and 5 encode uncharacterized gene products, Hs.121554 (Group IA), Hs.178062 (Group IA), Hs.245431 (Group IB), Hs.97643 (Group IC) and Hs.195932 (Group IIA). With regard to the presence of SAGE tags corresponding to known gene product ADAM2/Hs.177959 in normal colon tissue (Group IC), our RT-PCR expression data provides no evidence for ADAM2/Hs.177959 expression in normal colon (FIG. 1A). In addition to the 7 testis-restricted transcripts, 3 other Unigene clusters were expressed in a limited number of normal tissues. Transcripts encoding Regulatory factor X4 (RFX4, Hs.183009, Group IA) were detected only in testis and brain (0/18 other normal tissues). Two uncharacterized transcripts, Hs.128836 (Group IC) and Hs.293317 (IIA) were expressed in testis, ovary, cervix and lung (0/16 other normal tissues) and testis, prostate, ovary, lung, breast and colon (0/14 other normal tissues), respectively.

Expression of CT-Associated Unigene Clusters in Cancer

All seven of the testis-restricted transcripts can be considered CT gene products based on the presence of identical sequences in tumor derived EST libraries (Group I Unigene clusters) or SAGE libraries (Group IIA Unigene clusters). To confirm this in silico expression profile, the tissue restricted transcripts defined in the current study were analyzed by RT-PCR using a panel of RNA samples derived from a variety of malignant tissues. As shown in FIG. 1B, 3 of the 7 testis-restricted transcripts, ADAM2/Hs. 177959, Hs.245431, and Hs.178062 were also expressed in tumor tissue, and represent newly defined CT genes. These CT gene products represent 1 known gene product and 2 uncharacterized transcripts. The known protein, ADAM2/Hs.177959, was expressed exclusively in testis and in 2/16 cases of renal cancer (Tables 3 and 4).

TABLE 4
Conventional RT-PCR analysis of mRNA expression
frequencies of newly defined Cancer/Testis (CT) genes
in normal and malignant tissues

| | CT genes | | |
|---|---|---|---|
| | Hs.177959/ | | |
| | CT15 | Hs.245431/ | Hs.178062/ |
| Tissues | (ADAM2) | CT16 | CT17 |
| Normal Tissues | Testis only | Testis only | Testis only |
| Melanoma | 0/18 | 4/18 | 0/18 |
| Lung Cancer | 0/18 | 7/18 | 0/18 |
| Colon Cancer | 0/9 | 1/9 | 0/9 |
| Breast Cancer | 0/18 | 1/18 | 1/18 |
| Renal Cancer | 2/16 | 7/16 | 4/16 |
| Ovarian Cancer | 0/4 | 0/4 | 0/4 |
| Melanoma Cell | 0/8 | 4/8 | 0/8 |
| lines² | | | |
| Other Tumor | 0/4 | SW1045, | 0/4 |
| Cell lines² | | LU-17 | |

¹Normal tissue RNA panel included brain, testis, kidney, liver, pancreas, placenta, small intestine, heart, prostate, adrenal gland, spleen, fetal brain, colon, stomach, lung, bladder, ovary, breast, cervix and skeletal muscle
²Melanoma cell lines included SK-MEL-28, -23, -19, -109, -37, -10, -30, and -139
³Additional tumor cell lines tested include SK-LU-14 and SK-LU-17 lung cancer cells, and SW1045 and Fuji sarcoma cells

In accordance with proposed nomenclature for CT antigens (21), ADAM2 was given the CT designation CT15. ADAM2/CT15 is a member of the metalloproteinase-like, disintegrin-like cysteine-rich domain family of sperm surface proteins involved in egg/sperm interactions (51). The nucleotide and amino acid sequences for CT15/Hs.177959 are set forth as SEQ ID NOs:18 and 19.

Another of the CT gene products identified in the current study, designated CT16, is an uncharacterized transcript represented by the Hs.245431 Unigene cluster. CT16/Hs.245431 was expressed in 4/18 melanomas, 7/18 lung cancers 1/18 breast cancers, 1/9 colon cancers and 7/16 renal cancers (Tables 3 and 4). It was also expressed in several tumor cell lines, including SK-LU-17 lung cancer, SW1045 sarcoma, and 4/8 melanoma cell lines (SK-MEL-19, -109, -37, and -10), but not in normal melanocytes. The CT16/Hs.245431 cDNA sequence consists of 763 nucleotides (Genbank Acc# BC009230), containing a complete open reading frame, which encodes a putative full-length protein of 110 amino acids. The predicted CT16/Hs.245431 amino acid sequence is 30%-40% identical to members of the CT antigen family, GAGE-A (15), and 40%-50% identical to the GAGE-B/PAGE-1 (52). The nucleotide and amino acid sequences of Hs.245431 are presented as SEQ ID Nos:20 and 21, respectively.

The third newly defined CT gene product, designated CT17, represents the Hs. 178062 Unigene cluster, and was expressed in 1/18 breast cancers and 4/16 renal cancers (Tables 3 5 and 4). The CT17/Hs.178062 cDNA sequence is composed of 877 nucleotides (SEQ ID NO:22; Genbank accession # AA470035), encoding a partial protein of 202 amino acids (SEQ ID NO:23), which is 30% identical to phosphatidylserine-specific phospholipase Al (53).

Expression of the remaining 4 testis-restricted gene products, TSPNY/Hs.97643 10 (SEQ ID NOs:24 and 25), TTY12/Hs.195932 (SEQ ID Nos: 26 and 27), Ubqln 3/Hs.189184 (SEQ ID Nos:28 and 29)and Hs.121554 (SEQ ID Nos:30 and 31) (Table 2), was not detected in tumor tissue.

Three gene products defined in the current study as being expressed in a limited number of normal tissues were also expressed in tumor tissue (Table 5).

TABLE 5
Conventional RT-PCR analysis of mRNA expression
frequencies of differentially expressed, non-CT genes in
normal and malignant tissues

| | Differentially Expressed Non-CT genes | | |
|---|---|---|---|
| | Hs.183009 | | Hs.293317 |
| Tissues | (RFX4) | Hs.128836 | (CT16.2) |
| Normal Tissues¹ | Testis, Brain | Testis, Ovary, | Testis, Ovary, |
| | | Lung, Cervix | Lung, Colon, |
| | | | Breast, Prostate |
| Melanoma | 0/18 | 0/18 | 9/18 |
| Lung Cancer | 0/18 | 7/18 | 14/18 |
| Colon Cancer | 1/9 | 1/9 | 3/9 |
| Breast Cancer | 0/18 | 2/18 | 6/18 |
| Renal Cancer | 0/16 | 2/16 | 14/16 |
| Ovarian Cancer | 0/4 | 2/4 | 2/4 |
| Melanoma Cell | 4/8 | 0/8 | 0/8 |
| lines² | | | |
| Other Tumor | 0/4 | LU-14 | SW1045, |
| Cell lines² | | | LU-17 |

¹Normal tissue RNA panel included brain, testis, kidney, liver, pancreas, placenta, small intestine, heart, prostate, adrenal gland, spleen, fetal brain, colon, stomach, lung, bladder, ovary, breast, cervix and skeletal muscle
²Melanoma cell lines included SK-MEL-28, -23, -19, -109, -37, -10, -30, and -139
³Additional tumor cell lines tested include SK-LU-14 and SK-LU-17 lung cancer cells, and SW1045 and Fuji sarcoma cells

The known gene, Regulatory factor X 4 (RFX4, Hs.183009), was expressed exclusively in testis and brain, and also in 1/9 colon cancers, and 4/8 melanoma cell lines (SK-MEL-19, -37, -10, and -30), but not in normal melanocytes (Tables 3 and 5). RFX4/Hs.183009 (SEQ ID NOs:32 and 33) is presented in the Unigene database as a translocation product in breast cancer involving the ubiquitously expressed estrogen receptor 1 gene located on chromosome 6 and a novel, RFX-like gene (RFX-4) on chromosome 12 (54).

A second differentially expressed transcript, represented by the Hs. 128836 Unigene cluster, was expressed in normal testis, ovary, cervix and lung, and also in 7/18 lung cancers, 2/4 ovarian cancers, 2/18 breast cancers, 1/9 colon cancers and 2/16 renal cancers (Tables 3 and 5). The cDNA sequence of Hs.128836 is composed of 558 nucleotides (SEQ ID NO:34) encoding a putative partial protein of 164 amino acids (SEQ ID NO:35) with no similarity to characterized proteins or known protein motifs.

A third differentially expressed transcript, represented by the Hs.293317 Unigene cluster, was expressed in normal testis, ovary, lung, breast, prostate and colon, and also in 9/18 melanomas, 14/18 lung cancers, 6/18 breast cancers, 14/16 renal cancers, 2/4 ovarian cancers, and 3/9 colon cancers (Tables 3 and 5). Transcripts were also detected in 2 tumor cell lines, SW1045 sarcoma and SK-LU-17 lung cancer, but not in 8 melanoma cell lines, although it was expressed in normal melanocytes. Hs.293317 is a novel cDNA sequence, composed of 549 nucleotides (GenBank # AW002915) having a complete open reading frame encoding a putative full length protein of 110 amino acids that is 89% identical to the newly defined CT gene, CT16/Hs.245431 described above. Based on the similarity with CT16/Hs.245431, Hs.293317 has been designated CT16.2. The contig for the gene identified by Unigene cluster Hs.293317 is presented as SEQ ID NO:36. The polypeptide translation of the contig is presented as SEQ ID NO:37.

Quantitative Analysis of Cancer/Testis Gene Expression

To further investigate the mRNA expression profiles of CT15, CT16 and CT17, quantitative real time RT-PCR was performed using an RNA panel derived from various normal tissues and tumor specimens. For comparison, prototype CT antigens, NY-ESO-1 (18) and MAGE-3 (55), were also analyzed in this manner. The normalized level of CT gene expression in normal tissues and cancer, relative to their expression level in testis, is given in Table 6.

TABLE 6
Quantitative analysis of mRNA encoding Cancer/Testis gene
products in normal and malignant tissues relative to testis

| | Expression Level of mRNA Transcripts Encoding CT Gene | | | | |
|---|---|---|---|---|---|
| | Products in Various Tissues Relative to mRNA in Normal | | | | |
| | Testis | | | | |
| | CT15/ | CT16/ | CT17/ | | |
| Tissue | ADAM2 | Hs.245431 | Hs.178062 | NY-ESO-1 | MAGE-3 |
| Brain | 0 | 0 | 0 | 3% | 0 |
| Kidney | 0 | 0.1% | 0 | 0 | 0 |
| Liver | 0 | 0.4% | 0 | 0 | 0 |
| Pancreas | 1.0% | 0 | 0 | 0 | 0 |
| Colon | 0 | 0 | 0 | 2% | 0 |
| Lung | 0 | 0 | 0 | 3% | 0 |
| Ovary | 0 | 0 | 0 | 52% | 0 |
| Tumor #1¹ | 2% | 310% | 289% | 14% | 8% |
| Tumor #2 | 0.8% | 6400% | 19% | 19% | 11% |
| Tumor #3 | 0.07% | 320% | 0 | 6% | 30% |
| ¹Expression CT15/ADAM2 was analyzed in 3 renal cancer specimens (Tumor #1,RCC1; tumor #2, RCC5; tumor #3, RCC6). Expression of CT16 was analyzed in 2 melanoma specimens (tumor #1, Mel-1; tumor #2 Mel-11) and a breast cancer specimen (tumor #3, HBR-297). Expression of CT17 was analyzed in a breast cancer (tumor #1, HBR-297), renal cancer (tumor #2, RCC5) and a melanoma specimen (tumor #3, Mel-1). | | | | | |
| # Expression of NY-ESO-1 was analyzed in two lung cancer specimens (tumor #1, LU356; tumor #2, LU339) and a renal cancer specimen (tumor #3, RCC1). Expression of MAGE-3 was analyzed in two lung cancer specimens (tumor #1, Mel-1; tumor #2,Mel-11) and a lung cancer specimen (tumor #3, LU356). | | | | | |

Overall, real time RT-PCR analyses revealed either no expression, or considerably lower levels (3% or less) of CT gene transcripts in normal, non-gametogenic tissues compared with normal testis. In normal tissues, CT15 expression was detected in pancreas at 1% of the level detected in testis. In the case of CT16 mRNA expression in normal tissues, transcripts were detected only in kidney and liver, at 0.1% and 0.4% of the level detected in testis, respectively. Expression of both CT17 and MAGE-3 mRNA was restricted to testis. In the case of NY-ESO-1, the expression level in normal brain, colon and lung was 3%, 2% and 3%, respectively, of the level detected in testis. NY-ESO-1 was also detected in normal ovary at 52% of the level detected in testis. The copy number of CT transcripts per μg of total RNA was also calculated based on these relative expression levels (Table 6) and a comparison with a standard curve of homologous cDNA of known copy number. The expression level of CT genes in testis showed wide variation, with CT15 having the highest copy number (445,000 copies/μg RNA), followed by CT16 (149,000 copies/μg RNA), NY-ESO-1 (31,300 copies/μg RNA), CT17 (16,100 copies/μg RNA) and MAGE-3 (15,060 copies/μg RNA).

The expression level of CT genes in tumor tissue was also analyzed by quantitative real time RT-PCR. In renal cancer specimens, RCC1, RCC5 and RCC6, the expression level of CT15 was 2%, 0.07% and 0.8% of the level detected in testis (Table 6), respectively. Both the RCC1 and RCC6 tumors were positive for CT15 expression by conventional RT-PCR, while RCC5 was negative (FIG. 1B). As shown in Table 6, the level of CT16 expression in two melanoma samples was 3.1 and 64 times the level, respectively. In a breast cancer specimen, the level of CT16 expression was 3.2 times the level detected in testis. These two melanoma samples, and the breast cancer sample, were positive for CT16 expression when analyzed by conventional RT-PCR (FIG. 1B). In a breast cancer specimen (HBR297) and renal cancer specimen (RCC5), the level of CT17 expression was 2.89 and 0.19 times the level detected in testis (Table 6), respectively, which is consistent with conventional RT-PCR results. The level of NY-ESO-1 expression in two lung cancer specimens was 14% and 19% of the level detected in testis (Table 6), respectively. In a renal cancer specimen, NY-ESO-1 was expressed at a level that was 6% of the level detected in testis. Finally, MAGE-3 expression in two melanoma specimens and a lung cancer specimen was 8%, 11% and 30% of the level detected in testis, respectively.

Discussion

Expressed sequence tag (EST) databases are a repository of the human transcriptome, containing a wealth of nucleic acid sequence information and mRNA expression data. An extension of the EST database is Unigene, which pools information from public domain sequencing projects, including, EST, Genbank, ORESTES, and human genome projects, and links this information to a number of relevant databases, e.g., those dedicated to scientific literature, the human genome, the proteome, single nucleotide polymorphisms, and gene mutations. In conjunction with the Cancer Genome Anatomy Project, the Unigene database also provides tools for analyzing EST data, including in silico serial analysis of gene expression (SAGE), gene expression profiling, and digital differential display. In view of the immunotherapeutic importance of CT antigens, i.e., that they represent promising target molecules for antigen-specific cancer vaccines, the current study mined the Unigene database for gene clusters containing ESTs derived exclusively from cancer and testis cDNA libraries.

The current bioinformatic analysis identified approximately 1300 different cancer/testis-associated Unigene clusters. Preliminary evidence in support of the approach used to search the Unigene database was provided by the presence of four known CT antigens, CT11p, GAGE 4, MAGEB1, and SAGE, among these 1300 cancer/testis-associated Unigene clusters identified in the current study. Conversely, this bioinformatic analysis failed to identify members of 9 other previously identified CT gene families cited in the literature. The reason for this is that the database search tool (X profiler) used in the current study does not cross-reference more than two groups of cDNA libraries. Unigene clusters corresponding to the CT antigens not identified in the present study contain ESTs derived from cDNA libraries outside of the two cross-referenced pools (normal testis and cancer). For example, NY-ESO-1, SSX-2/HOM-MEL-40, and CT-7 Unigene clusters contain ESTs from placenta; BAGE, SCP-1, and CT-10 Unigene clusters contain ESTs from cell lines; the BRDT/CT9 Unigene cluster contains an EST from a subtracted testis library; and the sp32/OY-TES-1 Unigene cluster contains ESTs from normal retina and fetal heart. Also, the CTAGE-1 Unigene cluster contains only normal testis ESTs, but not tumor-derived ESTs.

The mRNA expression patterns of 73 of these cancer/testis-associated Unigene clusters were examined by RT-PCR using a panel of RNA samples derived from various normal and malignant tissues. Three of the 73 gene products, CT15/Hs.177959, CT16/Hs.245431, and CT17/Hs.178062, were shown by conventional RT-PCR to be expressed exclusively in testis and malignant tissues, and therefore have expression profiles analogous to CT antigens. Other similarities exist between the newly defined CT genes and known CT antigens. Two of the identified CT genes, CT16/Hs.245431 and CT17/Hs.178062, represent Unigene clusters that contain ESTs from testis, as well as melanoma and sarcoma cDNA libraries, respectively. These two tumor types are known to express a large proportion of the known CT antigens (56). Also, CT16/Hs.245431 maps to chromosome X, the site in the genome were 8 of the 14 known CT antigens map. Furthermore, the frequency of mRNA expression of the newly defined CT genes in cancer is consistent with those of previously defined CT antigens (20% -40% of a given tumor type, ref. 21), ranging from 11% -44% in the case of CT16 expression in colon cancer and renal cancer, respectively, and 5% -25% in the case of CT17 expression in breast cancer and renal cancer, respectively. Conversely, the apparent restricted nature of CT15/ADAM2 expression in normal testis and renal cancer is unique among CT genes. However, a relatively small sample size was examined in the current study, and a much broader mRNA expression may provide a more definitive conclusion regarding their expression frequencies in cancer.

With the exception of a proacrosin binding protein, OY-TES-1 (30), and synaptonemal complex protein-1 (20), the biological functions of CT antigens are not known. In the current study, two of the identified CT gene products encode proteins with known functions or functional motifs. ADAM2/CT15/Hs.177959, is a member of the metalloproteinase-like, disintegrin-like cysteine-rich domain family of cell surface proteases/adhesion molecules, and is believed to be involved egg/sperm membrane interactions (51). Although ADAM2/CT15 lacks a functional metalloproteinase domain it does contain a disintegrin domain, which may bind to integrin α₆β₁, or other similar molecules (57). Another CT gene product, CT17/Hs.178062 has similarity with phospholipases, which during fertilization play a role in sperm acrosomal exocytosis (58).

The remaining CT gene, CT16/Hs.245431, and its relative CT16.2/Hs.293317, are 30-50% similar to GAGE proteins (15, 52). Based on the similarities among the GAGE A family (90% or greater amino acid identity), and between the GAGE-A and GAGE-B families (40-50% amino acid identity), it was concluded that Hs.245431/CT16 represents a member of a new GAGE gene family, tentatively termed the CT 16 family, which also includes the tissue-restricted gene product, CT16.2/Hs.293317. The biological functions of GAGE proteins are not known, and few immunological responses to GAGE proteins have been reported. With the exception of GAGE-A1 and CT16, the majority of GAGE genes, including CT16.2, are expressed in a narrow range of normal adult tissues (59). Given the similarity among members of individual GAGE gene families, it is possible that the lack of an immune response to GAGE proteins reflects tolerance to highly similar, and more universally expressed GAGE genes.

In addition to CT genes, the current study also identified three highly tissue restricted gene products, RFX4/Hs.183009, Hs.128836, and Hs.293317, which are also expressed in cancer. RFX4 was expressed only in normal testis and brain, as well as in 1/9 colon cancers and 4/8 melanoma cell lines. RFX4 can therefore be considered a putative member of a group of proteins, termed cancer/testis/brain antigens (CTB antigens). Other CTB antigens include CDR (60), Ma1(61), and Ma2 (62), which were identified by Posner and colleagues as the target molecules recognized by autoantibodies in patients with paraneoplastic syndromes. RFX4 belongs to a family of DNA binding proteins that regulate transcription of MHC class II genes (63). Defects in genes encoding RFX proteins, such as RFXANK, RFX5 and RFXAP, lead to the development of Bare Lymphocyte Syndrome, a severe autosomal recessive immunodeficiency disease (reviewed in 64). Given the down-regulated expression of MHC genes in testis, brain and cancer, expression of RFX genes in these tissues may be of significance. Two uncharacterized transcripts, Hs.128836 and Hs.293317, also had mRNA expression profiles restricted to a limited number of normal tissues and cancer. Due to a lack of functional domains, the biological significance of these gene products remains to be determined.

In addition to the 3 CT genes and 3 tissue restricted transcripts, 4 other gene products having testis-restricted expression profiles were identified, including Hs.121554, Hs.97643, Hs.195932, and Hs.189184. Unigene clusters corresponding to these 4 testis restricted gene products also contain ESTs and/or SAGE tags derived from tumor tissue.

Continued expression analysis of these gene products, using enlarged panels of RNA derived from a wider variety of malignant tissues, may lead to their detection in tumor tissue and subsequent classification as CT genes. With regard to the remaining 1200 cancer/testis-associated Unigene clusters which were not examined by RT-PCR, further study will focus on those gene products having in silico expression profiles corresponding to CT-related (Group I) Unigene clusters and testis-related Unigene clusters with SAGE tags derived from tumor tissues (Group IIA). A method described by Loging and colleagues for rapid expression screening by real-time RT-PCR should advance these studies (65).

The use of individual CT gene products as target molecules for generic cancer vaccines may be inadequate based on their relatively low expression frequencies among cancer patient populations, heterogeneous expression within the tumor itself and antigen loss by a given tumor. An alternative is the development of polyvalent cancer vaccine containing epitopes encoded by many different CT genes. Such polyvalent vaccines would be an effective way to increase the number of cancer patients eligible for vaccination and may also overcome some of the obstacles associated with tumor heterogeneity and immune escape. To this end, the current study added CT15, CT16 and CT17 to the repertoire of proteins available for polyvalent CT cancer vaccines. Furthermore, ADAM2/CT15 can be considered a target molecule with dual immunotherapeutic value, since its cell surface localization makes it a potential target for monoclonal antibody based immunotherapies as well. In conclusion, the Unigene database contains a wealth of information, that when tapped into, can lead to the discovery of new cancer-related genes of therapeutic significance.

### Example 3

### Confirming the Identity of the CT Antigens

The length of the sequences identified above can be extended, providing additional sequence regions with which to search for related sequences in the gene databases. Elongation of the sequences described above is done using standard methods, (e.g. PCR) to extend the DNA sequences beyond the regions currently known, particularly for those sequences that encode an apparently incomplete protein. PCR-based amplification methods include 5′ RACE, which allows the isolation of the missing 5′ ends of the known, partial cDNAs. In addition, 3′ RACE is also used to extend the missing 3′ ends of the cDNAs. These additional end regions are sequenced, and the information used to screen the databases for matches and homologies.

Another method for lengthening the known sequences is through traditional library screening procedures, which allow isolation of longer sequences from libraries. Once extended sequences are identified, they are used to search the gene databases for sequence matches and the subsequent examination of expression patterns. The libraries used in the screening procedures are general libraries, or more tissue-specific or developmental stage-specific libraries.

### Example 4

### Preparation of Recombinant CT Antigens

CT15, CT16 and CT17 were expressed as his-tagged proteins in E. coli using histidine-tag-containing vector pQE30 (Qiagen, Valencia, Calif.) as described in ref. 40. The induction of recombinant protein synthesis and subsequent purification by Ni⁺² column were performed as described (Chen et al., Proc. Natl. Acad. Sci. USA. 91:1004-1008, 1994).

In alternative methods, the clones encoding CT antigens are subcloned into a baculovirus expression vector, and the recombinant expression vectors are introduced into appropriate insect cells. Baculovirus/insect cloning systems are preferred because post-translational modifications are carried out in the insect cells. Another preferred eukaryotic system is the Drosophila Expression System from Invitrogen. Clones which express high amounts of the recombinant protein are selected and used to produce the recombinant proteins. Other systems, including yeast expression systems and mammalian cell culture systems also can be used.

### Example 5

### Preparation of Antibodies to CT Antigens

The recombinant CT antigens produced as in Example 4 above are used to generate polyclonal antisera and monoclonal antibodies according to standard procedures. The antisera and antibodies so produced are tested for correct recognition of the CT antigens by using the antisera/antibodies in assays of cell extracts of patients known to express the particular CT antigen (e.g. an ELISA assay). These antibodies can be used for experimental purposes (e.g. localization of the CT antigens, immunoprecipitations, Western blots, etc.) as well as diagnostic purposes (e.g., testing extracts of tissue biopsies, testing for the presence of CT antigens).

The antibodies are useful for accurate and simple typing of cancer tissue samples for expression of the CT antigens.

### Example 6

### Expression of CT Antigens in Cancers of Similar and Different Origin

The expression of one or more of the CT antigens is tested in a range of tumor samples to determine which, if any, other malignancies should be diagnosed and/or treated by the methods described herein. Tumor cell lines and tumor samples are tested for CT antigen expression, preferably by RT-PCR or real time PCR according to the procedures described above. Northern blots also are used to test the expression of the CT antigens. Antibody based assays, such as ELISA and western blot, also can be used to determine protein expression. A preferred method of testing expression of CT antigens (in other cancers and in additional same type cancer patients) is allogeneic serotyping using a modified SEREX protocol.

In all of the foregoing, extracts from the tumors of patients who provided sera for the initial isolation of the CT antigens are used as positive controls. The cells containing recombinant expression vectors described in the Examples above also can be used as positive controls.

The results generated from the foregoing experiments provide panels of multiple cancer associated nucleic acids and/or polypeptides for use in diagnostic (e.g. determining the existence of cancer, determining the prognosis of a patient undergoing therapy, etc.) and therapeutic methods (e.g., vaccine composition, etc.).

### Example 7

### HLA Typing of Patients Positive for CT Antigens

To determine which HLA molecules present peptides derived from the CT antigens of the invention, cells of the patients which express the CT antigens are HLA typed. Peripheral blood lymphocytes are taken from the patient and typed for HLA class I or class II, as well as for the particular subtype of class I or class II. Tumor biopsy samples also can be used for typing. HLA typing can be carried out by any of the standard methods in the art of clinical immunology, such as by recognition by specific monoclonal antibodies, or by HLA allele-specific PCR (e.g. as described in WO97/31126).

### Example 8

### Characterization of CT Antigen Peptides Presented by MHC Class I and Class II Molecules

Antigens which provoke an antibody response in a subject may also provoke a cell-mediated immune response. Cells process proteins into peptides for presentation on MHC class I or class II molecules on the cell surface for immune surveillance. Peptides presented by certain MHC/HLA molecules generally conform to motifs. These motifs are known in some cases, and can be used to screen the CT antigens for the presence of potential class I and/or class II peptides. Summaries of class I and class II motifs have been published (e.g., Rammensee et al., Immunogenetics 41:178-228, 1995). Based on the results of experiments such as those described above, the HLA types which present the individual CT antigens are known. Motifs of peptides presented by these HLA molecules thus are preferentially searched.

One also can search for class I and class II motifs using computer algorithms. For example, computer programs for predicting potential CTL epitopes based on known class I motifs has been described (see, e.g., Parker et al, J. Immunol. 152:163, 1994; D'Amaro et al., Human Immunol. 43:13-18, 1995; Drijfhout et al., Human Immunol. 43:1-12, 1995). Computer programs for predicting potential T cell epitopes based on known class II motifs has also been described (see, e.g. Sturniolo et al., Nat Biotechnol 17(6):555-61, 1999). HLA binding predictions can conveniently be made using an algorithm available via the Internet on the National Institutes of Health World Wide Web site at URL http://bimas.dcrt.nih.gov. See also the website of: SYFPEITHI: An Internet Database for MHC Ligands and Peptide Motifs (access via http://www.uni-tuebingen.de/uni/kxi/ or http://134.2.96.221/scripts/hlaserver.dll/EpPredict.htm. Methods for determining HLA class II peptides and making substitutions thereto are also known (e.g. Strominger and Wucherpfennig (PCT/US96/03182)).

### Example 9

### Identification of the Portion of a Cancer Associated Polypeptide Encoding an Antigen

To determine if the CT antigens identified and isolated as described above can provoke a cytolytic T lymphocyte response, the following method is performed. CTL clones are generated by stimulating the peripheral blood lymphocytes (PBLs) of a patient with autologous normal cells transfected with one of the clones encoding a CT antigen polypeptide or with irradiated PBLs loaded with synthetic peptides corresponding to the putative protein and matching the consensus for the appropriate HLA class I molecule (as described above) to localize an antigenic peptide within the CT antigen clone (see, e.g., Knuth et al., Proc. Natl. Acad. Sci. USA 81:3511-3515, 1984; van der Bruggen et al., Eur. J. Immunol. 24:3038-3043, 1994). These CTL clones are screened for specificity against COS cells transfected with the CT antigen clone and autologous HLA alleles as described by Brichard et al. (Eur. J Immunol. 26:224-230, 1996). CTL recognition of a CT antigen is determined by measuring release of TNF from the cytolytic T lymphocyte or by ⁵¹Cr release assay (Herin et al., Int. J. Cancer 39:390-396, 1987). If a CTL clone specifically recognizes a transfected COS cell, then shorter fragments of the CT antigen clone transfected in that COS cell are tested to identify the region of the gene that encodes the peptide. Fragments of the CT antigen clone are prepared by exonuclease III digestion or other standard molecular biology methods. Synthetic peptides are prepared to confirm the exact sequence of the antigen.

Optionally, shorter fragments of CT antigen cDNAs are generated by PCR. Shorter fragments are used to provoke TNF release or ⁵¹Cr release as above.

Synthetic peptides corresponding to portions of the shortest fragment of the CT antigen clone which provokes TNF release are prepared. Progressively shorter peptides are synthesized to determine the optimal CT antigen tumor rejection antigen peptides for a given HLA molecule.

A similar method is performed to determine if the CT antigen contains one or more HLA class II peptides recognized by T cells. One can search the sequence of the CT antigen polypeptides for HLA class II motifs as described above. In contrast to class I peptides, class II peptides are presented by a limited number of cell types. Thus for these experiments, dendritic cells or B cell clones which express HLA class II molecules preferably are used.

### REFERENCES

1. Boyse E A, Miyazawa M, Aoki T, Old L J. Ly-A and Ly-B: two systems of lymphocyte isoantigens in the mouse. Proc Royal Soc Brit 1968; 170: 175-193. (PMID:4385242)

2. Boyse E A, Old L J. Some aspects of normal and abnormal cell surface genetics. Ann Rev Genet 1969; 3: 269-290.

3. DeLeo A B, Jay G, Appella E, Dubois G C, Law L W, Old L J. Detection of a transformation-related antigen in chemically induced sarcomas and other transformed cells of the mouse. Proc Natl Acad Sci USA 1979; 76: 2420-2424. (PMID: 221923)

4. Lilly F, Boyse E A, Old L J. Genetic basis of susceptibility to viral leukemogenesis. Lancet 1965; ii:1207-1209.

5. Carswell E A, Old L J, Kassel R L, Green S, Fiore N C, Williamson B. An endotoxin-induced serum factor that causes necrosis of tumors. Proc Natl Acad Sci USA 1975; 72: 3666-3670. (PMID: 1103152)

6. Traversari C, van der Bruggen P, Van den Eynde B, Hainaut P, Lemoine C, Ohta N, Old L J, Boon T. Transfection and expression of a gene coding for a human melanoma antigen recognized by autologous cytolytic T lymphocytes. Immunogenetics 1992; 35: 145-152. (PMID: 1537606)

7. van der Bruggen P, Traversari C, Chomez P, Lurquin C, De Plaen E, Van den Eynde B, Knuth A, and Boon, T, A gene encoding an antigen recognized by cytolytic T lymphocytes on a human melanoma. Science 1991; 254:1643-1647. (PMID: 1840703)

8. Old L J. Cancer immunology: The search for specificity. Cancer Res 1981; 41: 361-375. (PMID: 7004632)

9. Knuth A, Danowski B, Oettgen H F, Old L J. T cell-mediated cytotoxicity against malignant melanoma: Analysis with IL-2-dependent T cell cultures. Proc Natl Acad Sci USA 1984; 81: 3511-3515. (PMID: 6610177)

10. Sahin, U, Türeci Ö, Schmitt, Cochlovius B, Johannes T, Schmits R, Stenner F, Luo G, Schobert I, Pfreundschuh M. Human neoplasms elicit multiple specific immune responses in the autologous host. Proc Natl Acad Sci USA 1995; 92:11810-11813. (PMID: 8524854)

11. Old L J, Chen Y T. New paths in human cancer serology. J Exp Med 1998; 187:1163-1167. (PMID: 9547328)

12. De Plaen E, Arden K, Traversari C, Gaforio J J, Szikora J P, De Smet C, Brasseur F, van der Bruggen P, Lethé B, Lurquin C, Brasseur R, Chomez P, De Backer O, Cavenee W, and Boon T. Structure, chromosomal localization and expression of twelve genes of the MAGE family. Immunogenetics 1994; 40: 360-369. (PMID: 7927540)

13. Muscatelli F, Walker AP, De Plaen E, Stafford A N, Monaco A P. Isolation and characterization of a new MAGE gene family in the Xp21.3 region. Proc Natl Acad Sci USA 1995; 92: 4987-4991. (PMID: 7761436)

14. Boël P, Wildmann C, Sensi M L, Brasseur R, Renauld J C, Coulie P, Boon T, van der Bruggen P. BAGE, a new gene encoding an antigen recognized on human melanomas by cytolytic T lymphocytes. Immunity 1995; 2: 167-175. (PMID: 7895173)

15. Van den Eynde B, Peeters O, De Backer O, Gaugler B, Lucas S, Boon T. A new family of genes coding for an antigen recognized by autologous cytolytic T lymphocytes on a human melanoma. J Exp Med 1995;182: 689-698. (PMID: 7544395)

16. De Backer 0, Arden K C , Boretti M, Vantomee V, De Smet C, Czekay S, Viars C S, De Plaen E, Brasseur F, Chomez P, Van den Eynde B, Boon T, van der Bruggen P. Characterization of the GAGE genes that are expressed in various human cancer and in normal testis. Cancer Res 1999; 59: 3157-65. (PMID: 10397259)

17. Güre A O, Türeci O, Sahin U, Tsang S, Scanlan M J, Jager E, Knuth A, Pfreundschuh M, Old L J, Chen Y T. SSX, a multigene family with several members transcribed in normal testis and human cancer. Int. J. Cancer 1997; 72: 965-971. (PMID: 9378559)

18. Chen Y T, Scanlan M J, Sahin U, Türeci O, Güre A O, Tsang S, Williamson B, Stockert E, Pfreundschuh M, Old L J. A testicular antigen aberrantly expressed in human cancers detected by autologous antibody screening. Proc. Natl. Acad. Sci USA 1997; 94:1914-1918. (PMID: 9050879)

19. Lethe B, Lucas S, Michaux L, De Smet C, Godelaine D, Serrano A, De Plaen E, Boon T. LAGE-1: a new gene with tumor specificity. Int J Cancer 1998;76:903-908. (PMID: 9626360)

20. Türeci Ö, Sahin U, Zwick C, Koslowski M, Seitz G, Pfreundschuh M. Identification of a meiosis-specific protein as a new member of the class of cancer/testis antigens. Proc Natl Acad Sci USA 1998; 95: 5211-5216. (PMID: 9560255)

21. Chen Y T, Güre A O, Tsang S, Stockert E, Jäger E, Knuth A, Old L J. Identification of multiple cancer/testis antigens by allogeneic antibody screening of a melanoma cell line library. Proc Natl Acad Sci USA 1998; 95: 6919-6923. (PMID: 9618514)

22. Lucas S, De Smet C, Arden K C, Viars C S, Lethe B, Lurquin C, Boon T. Identification of a new MAGE gene with tumor-specific expression by representational difference analysis. Cancer Res 1998; 58: 743-752. (PMID: 9485030)

23. Sahin U, Koslowski M., Türeci Ö, Eberle T, Zwick C, Romeike B, Moringlane J R, Schwechheimer K, Feiden W., Pfreundschuh M. Expression of cancer/testis genes in human brain tumors. Clin Cancer Res 2000;10: 3916-3922. (PMID: 11051238)

24. Scanlan M J, Altorki NK, Güre A O, Williamson B, Jungbluth A, Chen Y T, Old L J. Expression of cancer-testis antigens in lung cancer: definition of bromodomain testis-specific gene (BRDT) as a new CT gene CT9. Cancer Lett. 2000; 150:155-164. (PMID: 10704737)

25. Güre A O, Stockert E, Arden K C, Boyer A D, Viars C S, Scanlan M J, Old L J, Chen Y T. CT10: a new cancer-testis (CT) antigen homologous to CT7 and the MAGE family, identified by representational difference analysis. Int. J. Cancer 2000; 85: 726-732. (PMID: 10699956)

26. Lucas S, De Plaen E, Boon T. MAGE-B5, MAGE-B6, MAGE-C2 and MAGE-C3: four new members of the MAGE family with tumor-specific expression. Int. J. Cancer 2000; 87:55-60. (PMID: 10861452)

27. Zendman A J, Cornelissen I M, Weidle U H, Ruiter D J, van Muijen G N. CTp11, a novel member of the family of human cancer/testis antigens. Cancer Res. 1999, 59: 6223-6239. (PMID: 10626816)

28. Martelange V, De Smet C, De Palen E, Lurquin C, Boon, T. Identification on a human sarcoma of two new genes with tumor-specific expression. Cancer Res 2000; 60: 3848-3855. (PMID: 10919659)

29. Eichmüller S, Usener D, Dummer R, Stein A, Thiel D, Schadendorf D. Serological detection of cutaneous T cell lymphoma-associated antigens. Proc Natl Acad Sci USA 2001; 98: 629-634. (PMID: 11149944)

30. Ono T, Kurashige T, Harada N, Noguchi Y, Saika T, Niikawa N, Aoe M, Nakamura S, Higashi T, Hiraki A, Wada H, Kumon H, Old L, Nakayama E. Identification of proacrosin binding protein sp32 precursor as a human cancer/testis antigen. Proc Natl Acad Sci. USA 2001; 98:3 282-3287. (PMID: 11248070)

31. Jungbluth A, Busam K, Kolb D, Iversen K, Coplan K, Chen Y T, Spagnoli G C, Old L J. Expression of MAGE-antigens in normal tissues and cancer. Int J Cancer. 2000; 85:460-5. (PMID: 10699915)

32. Jungbluth A, Busam K, Iversen, K, Kolb D, Coplan K, Chen Y T, Stockert E, Zhang P, Old, L J. Cancer-Testis (CT) antigens MAGE-1, MAGE-3, NY-ESO-1, and CT7 are expressed in female germ cells. Mod Path. In press 2001.

33. Jungbluth A, Iversen K, Kolb D, Coplan K, Chen Y T, Stockert E, Old L J, Vogel M. Expression of CT (Cancer/Testis) antigens MAGE, NY-ESO-1, and CT7 in placenta. German Soc for Path. Submitted 2001.

34. Jungbluth A, Stockert E, Chen YT, Kolb D, Iversen K, Coplan K, Williamson B, Altorki N, Busam K J, Old L J. Monoclonal antibody MA454 reveals a heterogeneous expression pattern of MAGE-1 antigen in formalin-fixed paraffin embedded lung tumours. British Journ Cancer 2000; 83: 493-497. (PMID: 10945497)

35. Meuwissen R J L, Offenberg, H H, Dietrich A J, Riesewijk A, van Iersel M, Heting C. A coiled-coil related protein specific for synapsed regions of meiotic prophase chromosomes. EMBO J 1992; 11: 5091-5100. (PMID: 1464329)

36. Baba T, Niida Y, Michikawa Y, Kashiwabara S, Kodaira K, Takenaka M, Kohno N, Gerton G L, Arai Y. An acrosomal protein, sp32, in mammalian sperm is a binding protein specific for two proacrosins and an acrosin intermediate. J Biochem 1994; 269:10133-10140. (PMID: 8144514)

37. Brasseur F, Rimoldi D, Liénard D, Lethe B, Carrel S, Arienti F, Suter L, Vanwijck R, Bourlond A, Humblet Y, Vacca A, Conese M, Lahaye T, Degiovanni G, Deraemaecker R, Beauduin M, Sastre X, Salamon E, Dréno B, Jager E, Knuth A, Chevreau C, Suciu S, Lachapelle J-M, Pouillart P, Parmiani G, Lejeune F, Cerottini J-C, Boon T, Marchand M. Expression of MAGE gene in primary and metastatic cutaneous melanoma. Int J Cancer 1995; 63 :375-380. (PMID: 7591235)

38. Patard J J, Brasseur F, Gil-Diez S, Radvanyi F, Marchand M, Francois P, Abi-Aad A, VanCangh P, Abbou C C, Chopin D, Boon T. Expression of MAGE genes in transitional-cell carcinomas of the urinary bladder. Int J Cancer 1995; 64:60-64. PMID: 7665250)

39. Kurashige T, Noguchi Y, Saika T, Ono T, Nagata Y, Jungluth A, Ritter G, Chen Y T, Stockert E, Tsushima T, Kumon H, Old L J, Nakayama E. NY-ESO-1 expression and immunogenicity associated with transitional cell carcinoma: correlation with tumor grade. Cancer Res. 2001; 61:4671-4674.

40. Stockert E., Jäger E, Chen YT, Scanlan M, Gout I, Karbach J, Arand M, Knuth A, Old, L J. A survey of the humoral response of cancer patients to a panel of human tumor antigens. J Exp Med 1998;187:1349-1354. (PMID: 9547346)

41. Jäger E., Nagata Y, Gnjatic S, Wada H, Stockert E, Karbach J, Dunbar P R, Lee S Y, Jungbluth A, Jäger D, Arand M, Ritter G, Cerundolo V, Dupont B, Chen Y T, Old L J, Knuth A. Monitoring CD8 T cell responses to NY-ESO-1: Correlation of humoral and cellular immune responses. Proc Natl Acad Sci USA 2000; 97: 4760-4765. (PMID: 10781081)

42. De Smet C, De Backer 0, Faraoni I, Lurquin C, Brasseur F, Boon T. The activation of human gene MAGE-1 in tumor cells is correlated with geneome-wide demethylation. Proc. Natl. Acad. Sci USA 1996; 93:7149-7153 (.PMID: 8692960)

43. Jungbluth A, Chen Y T, Stockert E, Busam K J, Kolb D, Iversen K, Coplan K, Williamson B, Altorki N, Old L J. Immunohistochemical analysis of NY-ESO-1 antigen expression in normal and malignant tumors. Int J Cancer. 2001; 92:856-860.

44. Jungbluth A, Antonescu C, Busam K, Iversen K, Kolb D, Coplan K, Chen Y T, Stockert E, Ladanyi M, Old, L J. Monophasic and biphasic synovial sarcomas abundantly express cancer/testis antigen NY-ESO-1, but not MAGE-A1 or CT7. Int J Cancer. 2001; 94(2):252-6.

45. Antonescu C, Busam K, Iversen K, Kolb D, Coplan K, Spagnoli G, Ladanyi M, Old L J, Jungbluth, A. MAGE antigen expression in monophasic and biphasic synovial sarcoma. Mod Path. Submitted 2001.

46. Beard J. The cancer problem. Lancet 1905;1:281-203.

47. Gurchot C. The trophoblast theory of cancer. Oncology 1975; 31: 310-333. (PMID: 1107920)

48. Iles R K, Chard T. Human Chorionic Gonadotropin Expression by Bladder Cancers: Biology and Clinical Potential. J Urol 1991; 145 :453-458. (PMID: 1705292)

49. Acevedo H F, Tong J Y, Hartsock R J. Human Chorionic Gonadotropin-Beta Subunit Gene Expression in Cultured Human Fetal and Cancer Cells of Different Types and Origins. Cancer 1995; 76: 1467-1475. (PMID: 8620425)

50. Dirnhofer S, Koessler P, Ensigner C, Feichtinger H, Madersbacher S, Berger P. Production of Trophoblastic Hormones by Transitional Cell Carcinoma of the Bladder: Association to Tumor Stage and Grade. Hum Path 1998; 29: 377-382. (PMID: 9563788)

51. Vidaeus C M, von Kapp-Herr C, Golden W L, Eddy R L, Shows T B, Herr J C. Human fertilin beta: identification, characterization, and chromosomal mapping of an ADAM gene family member. Mol Reprod Dev. 1997;46:363-9.

52. Brinkmann U, Vasmatzis G, Lee B, Yerushalmi N, Essand M, Pastan I. PAGE-1, an X chromosome-linked GAGE-like gene that is expressed in normal and neoplastic prostate, testis, and uterus. Proc. Natl. Acad. Sci. USA 1998;95: 10757-62.

53. Nagai Y, Aoki J, Sato T, Amano K, Matsuda Y, Arai H, et al. An alternative splicing form of phosphatidylserine-specific phospholipase A1 that exhibits lysophosphatidylserine-specific lysophospholipase activity in humans. J. Biol. Chem. 1999;274:11053-9.

54. Dotzlaw H, Alkhalaf M, Murphy L C. Characterization of estrogen receptor variant mRNAs from human breast cancers. Mol Endocrinol. 1992;6:773-85.

55. Gaugler B, Van den Eynde B, van der Bruggen P, Romero P, Gaforio J J, De Plaen E, et al. Human gene MAGE-3 codes for an antigen recognized on a melanoma by autologous cytolytic T lymphocytes. J. Exp. Med. 1994; 179:921-30.

56. Chen Y T, Scanlan M J, Obata Y, Old L J. Identification of human tumor antigens by serological expression cloning. (2000) In: Rosenberg S A. Principles and Practice of Biologic Therapy of Cancer. Philadelphia: Lippincott Williams & Wilkins. 2000:557-70.

57. Primakoff P, Myles D G. The ADAM gene family: surface proteins with adhesion and protease activity. Trends Genet. 2000;16:83-7.

58. Roldan E R. Role of phospholipases during sperm acrosomal exocytosis. Front Biosci. 1998; 3:D1109-19.

59. Scarcella D L, Chow C W, Gonzales M F, Economou C, Brasseur F, Ashley D M. Expression of MAGE and GAGE in high-grade brain tumors: a potential target for specific immunotherapy and diagnostic markers. Clin Cancer Res. 1999; 5:335-41.

60. Dropcho E J, Chen Y T, Posner J B, Old L J. Cloning of a brain protein identified by autoantibodies from a patient with paraneoplastic cerebellar degeneration. Proc Natl Acad Sci USA. 1987;84:4552-56.

61. Dalmau J, Gultekin S H, Voltz R, Hoard R, DesChamps T, Balmaceda C, et al. Mal, a novel neuron- and testis-specific protein, is recognized by the serum of patients with paraneoplastic neurological disorders. Brain. 1999; 122: 27-39.

62. Voltz R, Gultekin S H, Rosenfeld M R, Gerstner E, Eichen J, Posner J B, et al. A serologic marker of paraneoplastic limbic and brain-stem encephalitis in patients with testicular cancer. N Engl J Med. 1999;340:1788-95.

63. Steimle V, Durand B, Barras E, Zufferey M, Hadam M R, Mach B, et al. A novel DNA-binding regulatory factor is mutated in primary MHC class II deficiency (bare lymphocyte syndrome). Genes Dev 1995;9:1021-32.

64. Mach B, Steimle V, Reith W. MHC class II-deficient combined immunodeficiency: a disease of gene regulation. Immunol Rev. 1994; 138:207-21.

65. Loging W T, Lal A, Siu I M, Loney T L, Wikstrand C J, Marra M A, et al. Identifying potential tumor markers and antigens by database mining and rapid expression screening. Genome Res. 2000; 10:1393-1402.

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method of diagnosing a cancer, comprising:
contacting a non-testis biological sample isolated from a subject with an agent that specifically binds to a nucleic acid molecule, an expression product thereof, or a fragment of the expression product thereof complexed with an HLA molecule, wherein the nucleic acid molecule comprises a nucleotide sequence selected from the group consisting of SEQ ID NOs:18, 20 and 22, and determining the interaction between the agent and the nucleic acid molecule or the expression product to diagnose the cancer in the subject.

2. The method of claim 1, wherein the agent is selected from the group consisting of
(a) a nucleic acid molecule comprising a nucleotide sequence selected from the group consisting of SEQ ID NOs:18, 20 and 22 or a fragment thereof,
(b) an antibody that binds to an expression product of a nucleic acid molecule comprising a nucleotide sequence selected from the group consisting of SEQ ID NOs:18, 20 and 22, and
(c) an agent that binds to a complex of an HLA molecule and a fragment of an expression product of a nucleic acid molecule comprising a nucleotide sequence selected from the group consisting of SEQ ID NOs:18, 20 and 22.

3. The method of claim 1, wherein the cancer is characterized by expression of a plurality of human CT antigen precursors and wherein the agent is a plurality of agents, each of which is specific for a different human CT antigen precursor, and wherein said plurality of agents is at least 2, at least 3, at least 4, at least 5, at least 6, at least 7, or at least 8, at least 9 or at least 10 such agents.

4. A method of diagnosing a cancer, comprising:
contacting a non-testis, non-brain biological sample isolated from a subject with an agent that specifically binds to a nucleic acid molecule, an expression product thereof, or a fragment of an expression product thereof complexed with an HLA molecule, wherein the nucleic acid molecule comprises a nucleotide sequence set forth as SEQ ID NO:32, and
determining the interaction between the agent and the nucleic acid molecule or the expression product to diagnose the cancer in the subject.

5. A method of diagnosing a cancer, comprising:
contacting a non-testis, non-ovary, non-cervix, non-lung biological sample isolated from a subject with an agent that specifically binds to a nucleic acid molecule, an expression product thereof, or a fragment of an expression product thereof complexed with an HLA molecule, wherein the nucleic acid molecule comprises a nucleotide sequence set forth as SEQ ID NO:34, and
determining the interaction between the agent and the nucleic acid molecule or the expression product to diagnose the cancer in the subject.

6. A method of diagnosing a cancer, comprising:
contacting a non-testis, non-ovary, non-lung, non-breast, non-prostate, non-colon biological sample isolated from a subject with an agent that specifically binds to a nucleic acid molecule, an expression product thereof, or a fragment of an expression product thereof complexed with an HLA molecule, wherein the nucleic acid molecule comprises a nucleotide sequence set forth as SEQ ID NO:36, and
determining the interaction between the agent and the nucleic acid molecule or the expression product to diagnose the cancer in the subject.

7. A method for determining regression, progression or onset of a cancer characterized by expression of abnormal levels of a protein encoded by a nucleic acid molecule comprising a nucleotide sequence selected from the group consisting of SEQ ID NOs:18, 20 and 22, comprising
monitoring a plurality of non-testis samples obtained at different times from a subject who has or is suspected of having the cancer, for a parameter selected from the group consisting of
(i) the protein,
(ii) a peptide derived from the protein,
(iii) an antibody which selectively binds the protein or peptide,
(iv) cytolytic T cells specific for a complex of the peptide derived from the protein and an MHC molecule, and
(v) a nucleic acid molecule comprising a nucleotide sequence selected from the group consisting of SEQ ID NOs:18, 20 and 22; and.
comparing the parameters from the plurality of samples to determine regression, progression or onset of the cancer.

8. The method of claim 7, wherein the sample is a body fluid, a body effusion, cell or a tissue.

9. The method of claim 7, wherein the step of monitoring comprises contacting the sample with a detectable agent selected from the group consisting of
(a) an antibody which selectively binds the protein of (i), or the peptide of (ii),
(b) a protein or peptide which binds the antibody of (iii),
(c) a cell which presents the complex of the peptide and MHC molecule of (iv), and
(d) at least one nucleic acid probe or primer that hybridizes to the nucleic acid molecule of (v) or its complement.

10. The method of claim 9, wherein the antibody, the protein, the peptide, the cell or the nucleic acid probe or primer is labeled with a radioactive label or an enzyme.

11. The method of claim 7, wherein the protein is a plurality of proteins, the parameter is a plurality of parameters, each of the plurality of parameters being specific for a different of the plurality of proteins, at least one of which is a CT antigen protein encoded by a nucleic acid molecule comprising a nucleotide sequence selected from the group consisting of SEQ ID NOs:18, 20 and 22.

12. A pharmaceutical preparation for a human subject comprising
an agent which when administered to the subject enriches selectively the presence of complexes of an HLA molecule and a human CT antigen peptide, and
a pharmaceutically acceptable carrier, wherein the human CT antigen peptide is a fragment of a human CT antigen encoded by a nucleic acid molecule comprising a nucleotide sequence selected from the group consisting of SEQ ID NOs:18, 20 and 22.

13. The pharmaceutical preparation of claim 12, wherein the agent comprises a plurality of agents, each of which enriches selectively in the subject complexes of an HLA molecule and a different human CT antigen peptide, wherein at least one of the human CT antigens is encoded by a nucleic acid molecule comprising a nucleotide sequence selected from the group consisting of SEQ ID NOs:18, 20 and 22.

14. The pharmaceutical preparation of claim 12, wherein the agent is selected from the group consisting of
(1) an isolated polypeptide comprising the human CT antigen peptide,
(2) an isolated nucleic acid operably linked to a promoter for expressing the isolated polypeptide,
(3) a host cell expressing the isolated polypeptide, and
(4) isolated complexes of the polypeptide, and an HLA molecule.

15. The pharmaceutical preparation of claims 12-14, further comprising an adjuvant.

16. The pharmaceutical preparation of claim 12, wherein the agent is a cell expressing an isolated polypeptide comprising the human CT antigen peptide, and wherein the cell is nonproliferative.

17. The pharmaceutical preparation of claim 12, wherein the agent is a cell expressing an isolated polypeptide comprising the human CT antigen peptide, and wherein the cell expresses an HLA molecule that binds the polypeptide.

18. The pharmaceutical preparation of claim 17, wherein the cell expresses one or both of the polypeptide and HLA molecule recombinantly.

19. The pharmaceutical preparation of claim 17, wherein the cell is nonproliferative.

20. A composition comprising
an isolated agent that binds selectively a polypeptide comprising an amino acid sequence selected from the group consisting of SEQ ID NOs:21, 23, 25, 27, 29, 31, 35 and 37.

21. The composition of matter of claim 20, wherein the agent is an antibody or an antigen-binding fragment thereof.

22. The composition of claim 21, wherein the antibody is a monoclonal antibody, a chimeric antibody or a humanized antibody.

23. A composition of matter comprising a conjugate of the agent of claim 20 or 21 and a therapeutic or diagnostic agent.

24. The composition of matter of claim 23, wherein the conjugate is of the agent and a therapeutic or diagnostic that is a toxin.

25. A pharmaceutical composition comprising
an isolated nucleic acid molecule comprising a nucleotide sequence selected from the group consisting of SEQ ID NOs:18, 20 and 22, and
a pharmaceutically acceptable carrier.

26. The pharmaceutical composition of claim 25, wherein the isolated nucleic acid molecule comprises at least two isolated nucleic acid molecules coding for two different polypeptides, each polypeptide comprising a different human CT antigen.

27. The pharmaceutical composition of claim 25 or 26 further comprising an expression vector with a promoter operably linked to the isolated nucleic acid molecule.

28. The pharmaceutical composition of claim 25 or 26 further comprising a host cell recombinantly expressing the isolated nucleic acid molecule.

29. A pharmaceutical composition comprising
an isolated polypeptide comprising a polypeptide encoded by a nucleic acid molecule comprising a nucleotide sequence selected from the group consisting of SEQ ID NOs:18, 20 and 22, and
a pharmaceutically acceptable carrier.

30. The pharmaceutical composition of claim 29, wherein the isolated polypeptide comprises at least two different polypeptides, each comprising a different human CT antigen.

31. The pharmaceutical composition of claim 29 or 30, further comprising an adjuvant.

32. A protein microarray comprising at least one polypeptide encoded by a nucleic acid molecule comprising a nucleotide sequence selected from the group consisting of SEQ ID NOs:18, 20 and 22, or an antigenic fragment of the polypeptide.

33. The microarray of claim 32, wherein the at least one polypeptide comprises an amino acid sequence selected from the group consisting of SEQ ID NOs:19, 21 and 23.

34. A protein microarray comprising at least one polypeptide encoded by a nucleic acid molecule comprising a nucleotide sequence selected from the group consisting of SEQ ID NOs:24, 26, 28 and 30, or an antigenic fragment of the polypeptide.

35. The microarray of claim 34, wherein the at least one polypeptide comprises an amino acid sequence selected from the group consisting of SEQ ID NOs:25, 27, 29 and 31.

36. A protein microarray comprising at least one polypeptide encoded by a nucleic acid molecule comprising a nucleotide sequence selected from the group consisting of SEQ ID NOs:32, 34 and 36, or an antigenic fragment of the polypeptide.

37. The microarray of claim 36, wherein the at least one polypeptide comprises an amino acid sequence selected from the group consisting of SEQ ID NOs:33, 35 and 37.

38. A protein microarray comprising a plurality of antibodies or antigen-binding fragments thereof that specifically bind at least one polypeptide encoded by a nucleic acid molecule comprising a nucleotide sequence selected from the group consisting of SEQ ID NOs:18, 20 and 22, or an antigenic fragment of the polypeptide.

39. The microarray of claim 38, wherein the polypeptide comprises an amino acid sequence selected from the group consisting of SEQ ID NOs:19, 21 and 23.

40. A protein microarray comprising a plurality of antibodies or antigen-binding fragments thereof that specifically bind at least one polypeptide encoded by a nucleic acid molecule comprising a nucleotide sequence selected from the group consisting of SEQ ID NOs:24, 26, 28 and 30, or an antigenic fragment of the polypeptide.

41. The microarray of claim 40, wherein the polypeptide comprises an amino acid sequence selected from the group consisting of SEQ ID NOs:25, 27, 29 and 31.

42. A protein microarray comprising a plurality of antibodies or antigen-binding fragments thereof that specifically bind at least one polypeptide encoded by a nucleic acid molecule comprising a nucleotide sequence selected from the group consisting of SEQ ID NOs:32, 34 and 36, or an antigenic fragment of the polypeptide.

43. The microarray of claim 42, wherein the polypeptide comprises an amino acid sequence selected from the group consisting of SEQ ID NOs:33, 35 and 37.

44. A nucleic acid microarray comprising at least one nucleic acid molecule comprising a nucleotide sequence selected from the group consisting of SEQ ID NOs:18, 20 and 22, or a fragment thereof of at least 20 nucleotides that selectively hybridizes to its complement in a biological sample.

45. A nucleic acid microarray comprising at least one nucleic acid molecule comprising a nucleotide sequence selected from the group consisting of SEQ ID NOs:24, 26, 28 and 30, or a fragment thereof of at least 20 nucleotides that selectively hybridizes to its complement in a biological sample.

46. A nucleic acid microarray comprising at least one nucleic acid molecule comprising a nucleotide sequence selected from the group consisting of SEQ ID NOs:32, 34 and 36, or a fragment thereof of at least 20 nucleotides that selectively hybridizes to its complement in a biological sample.

47. An isolated fragment of a human CT antigen which, or a portion of which, binds a HLA molecule or a human antibody, wherein the CT antigen is encoded by a nucleic acid molecule comprising a nucleotide sequence selected from the group consisting of SEQ ID NOs:18, 20 and 22.

48. The fragment of claim 47, wherein the fragment is part of a complex with the HLA molecule.

49. The fragment of claim 47, wherein the fragment is between 8 and 12 amino acids in length.

50. A kit for detecting the expression of two or more human CT antigens comprising
two or more pairs of isolated nucleic acid molecules, each of which consists essentially of a nucleic acid molecule selected from the group consisting of (a) a 12-32 nucleotide contiguous segment of the nucleotide sequence of any of SEQ ID NOs:18, 20 or 22, and (b) complements of (a), wherein the contiguous segments are nonoverlapping, and wherein the nucleic acid molecules in each of the pairs are specific for a human CT antigen.

51. The kit of claim 50, wherein the pair of isolated nucleic acid molecules is constructed and arranged to selectively amplify at least a fragment of an isolated nucleic acid molecule selected from the group consisting of SEQ ID NOs:18, 20 and 22.

52. A method for treating a subject with a cancer characterized by expression of a human CT antigen, comprising
administering to the subject an amount of an agent, which enriches selectively in the subject the presence of complexes of a HLA molecule and a human CT antigen peptide, effective to ameliorate the disorder, wherein the human CT antigen peptide is a fragment of a human CT antigen encoded by a nucleic acid molecule comprising a nucleotide sequence selected from the group consisting of SEQ ID NOs:18, 20 and 22.

53. The method of claim 52, wherein the cancer is characterized by expression of a plurality of human CT antigens and wherein the agent is a plurality of agents, each of which enriches selectively in the subject the presence of complexes of an HLA molecule and a different human CT antigen peptide, wherein at least one of the human CT antigens is encoded by a nucleic acid molecule comprising a nucleotide sequence selected from the group consisting of SEQ ID NOs:18, 20 and 22.

54. The method of claim 52, wherein the agent is an isolated polypeptide encoded by a nucleic acid molecule comprising a nucleotide sequence selected from the group consisting of SEQ ID NOs:18, 20 and 22.

55. A method for treating a subject having a cancer characterized by expression of a human CT antigen in cells of the subject, comprising:
(i) removing an immunoreactive cell containing sample from the subject,
(ii) contacting the immunoreactive cell containing sample to the host cell under conditions favoring production of cytolytic T cells against a human CT antigen peptide that is a fragment of the human CT antigen,
(iii) introducing the cytolytic T cells to the subject in an amount effective to lyse cells which express the human CT antigen, wherein the host cell is transformed or transfected with an expression vector comprising an isolated nucleic acid molecule operably linked to a promoter, wherein the isolated nucleic acid molecule comprises a nucleotide sequence selected from the group consisting of SEQ ID NOs:18, 20 and 22.

56. The method of claim 55, wherein the host cell recombinantly expresses an HLA molecule which binds the human CT antigen peptide.

57. The method of claim 55, wherein the host cell endogenously expresses an HLA molecule which binds the human CT antigen peptide.

58. A method for treating a subject having a cancer characterized by expression of a human CT antigen in cells of the subject, comprising:
(i) identifying a nucleic acid molecule expressed by the cells of the cancer, wherein the nucleic acid molecule comprises a nucleotide sequence selected from the group consisting of SEQ ID NOs:18, 20 and 22, or a fragment thereof;
(ii) transfecting a host cell with a nucleic acid molecule selected from the group consisting of (a) the nucleic acid molecule identified, (b) a fragment of the nucleic acid identified which includes a segment coding for a human CT antigen, (c) degenerates of (a) or (b);
(iii) culturing said transfected host cells to express the transfected nucleic acid molecule, and;
(iv) introducing an amount of said host cells or an extract thereof to the subject effective to increase an immune response against the cells of the subject associated with the condition.

59. The method of claim 58, further comprising identifying an MHC molecule which presents a portion of an expression product of the nucleic acid molecule, wherein the host cell expresses the same MHC molecule as identified and wherein the host cell presents an MHC binding portion of the expression product of the nucleic acid molecule.

60. The method of claim 58, wherein the immune response comprises a B-cell response or a T cell response.

61. The method of claim 60, wherein the response is a T-cell response which comprises generation of cytolytic T-cells specific for the host cells presenting the portion of the expression product of the nucleic acid molecule or cells of the subject expressing the human CT antigen.

62. The method of claim 58, wherein the nucleic acid molecule is selected from the group consisting of SEQ ID NOs:18, 20 and 22.

63. The method of claim 58 or 59, further comprising treating the host cells to render them non-proliferative.

64. A method for treating or diagnosing or monitoring a subject having a cancer characterized by expression of a protein encoded by a nucleic acid molecule comprising a nucleotide sequence selected from the group consisting of SEQ ID NOs:18, 20, and 22, in cells or tissues other than testis, comprising
administering to the subject an antibody which specifically binds to the protein or a peptide derived therefrom, the antibody being coupled to a therapeutically or diagnostically useful agent, in an amount effective to treat, diagnose or monitor the condition.

65. The method of claim 64, wherein the antibody is a monoclonal antibody or an antigen-binding fragment thereof.

66. The method of claim 65, wherein the monoclonal antibody is a chimeric antibody or a humanized antibody.

67. A method for treating a cancer characterized by expression of a protein encoded by a nucleic acid molecule comprising a nucleotide sequence selected from the group consisting of SEQ ID NOs:18, 20 and 22, in cells or tissues other than testis, comprising
administering to a subject a pharmaceutical composition of any one of claims 12-19 and 25-31 in an amount effective to prevent, delay the onset of, or inhibit the condition in the subject.

68. The method of claim 67, further comprising first identifying that the subject expresses abnormal amounts of the protein in a non-testis tissue.

69. A method for treating a subject having a cancer characterized by expression of a protein encoded by a nucleic acid molecule comprising a nucleotide sequence selected from the group consisting of SEQ ID NOs:18, 20 and 22, in cells or tissues other than testis, comprising
(i) identifying cells from the subject which express abnormal amounts of the protein;
(ii) isolating a sample of the cells;
(iii) cultivating the cells, and
(iv) introducing the cells to the subject in an amount effective to provoke an immune response against the cells.

70. The method of claim 69, further comprising rendering the cells non-proliferative, prior to introducing them to the subject.

71. A method for treating a pathological cell condition characterized by expression of a protein encoded by a nucleic acid molecule comprising a nucleotide sequence selected from the group consisting of SEQ ID NOs:18, 20 and 22, in cells or tissues other than testis, comprising
administering to a subject in need thereof an effective amount of an agent which inhibits the expression or activity of the protein.

72. The method of claim 71, wherein the agent is an inhibiting antibody which selectively binds to the protein and wherein the antibody is a monoclonal antibody, a chimeric antibody, a humanized antibody or an antibody fragment.

73. The method of claim 71, wherein the agent is an antisense nucleic acid molecule which selectively binds to the nucleic acid molecule which encodes the protein.

74. A composition of matter useful in stimulating an immune response to a plurality of a proteins comprising an amino acid sequence selected from the group consisting of SEQ ID NOs: 19, 21 and 23 comprising
a plurality of peptides that are fragments of the proteins, wherein the peptides bind to one or more MHC molecules presented on the surface of non-testis cells.

75. The composition of matter of claim 74, wherein at least a portion of the plurality of peptides bind to MHC molecules and elicit a cytolytic response thereto.

76. The composition of matter of claim 74, wherein at least one of the proteins is encoded by a nucleic acid molecule comprising a nucleotide sequence selected from the group consisting of SEQ ID NOs:18, 20 and 22.

77. The composition of matter of claim 74, further comprising an adjuvant.

78. The composition of matter of claim 77, wherein said adjuvant is selected from the group consisting of saponins, GM-CSF, interleukins, and immunostimulatory oligonucleotides.

79. An isolated antibody which selectively binds to a complex of:
(i) a peptide that is a fragment of a protein comprising an amino acid sequence selected from the group consisting of SEQ ID NOs:19, 21 and 23, and
(ii) a MHC molecule to which binds the peptide to form the complex, wherein the isolated antibody does not bind to (i) or (ii) alone.

80. The antibody of claim 79, wherein the antibody is a monoclonal antibody, a chimeric antibody, a humanized antibody, or an antigen-binding fragment thereof.

81. A method for treating or diagnosing or monitoring a subject having a cancer characterized by expression of a protein encoded by a nucleic acid molecule comprising a nucleotide sequence selected from the group consisting of SEQ ID NOs:18, 20, and 22, in cells or tissues other than testis, comprising
administering to the subject the antibody of claim 79, the antibody being coupled to a therapeutically or diagnostically useful agent, in an amount effective to treat, diagnose or monitor the condition.

82. A method for treating or diagnosing or monitoring a subject having a cancer characterized by expression of a protein encoded by a nucleic acid molecule comprising a nucleotide sequence selected from the group consisting of SEQ ID NOs:18, 20, and 22, in cells or tissues other than testis, comprising
administering to the subject the antibody of claim 80, the antibody being coupled to a therapeutically or diagnostically useful agent, in an amount effective to treat, diagnose or monitor the condition.
